(19) United States
(12) Patent Application Publication
(10) Pub. No.: US 2024/0368251 A1
(43) Pub. Date: Nov. 7, 2024

(54) METHODS OF PROCESSING COLLAGEN FROM ANIMAL TISSUE

(71) Applicant: Grande Delta Corporation, Alameda, CA (US)

(72) Inventors: Allan Arthur TORNEY, Brampton (CA); Neil WILLCOCKS, Highland Park, IL (US); Lawrence JOHNSON, Alameda, CA (US); Gordon GREGORY, Alameda, CA (US); Amit INBART, Beit Elazari (IL); David GRAINGER, Salt Lake City, UT (US)

(21) Appl. No.: 18/568,728

(22) PCT Filed: Jun. 14, 2022
(86) PCT No.: PCT/US22/33374

§ 371 Date: Dec. 8, 2023

Related U.S. Application Data
(60) Provisional application No. 63/210,483, filed on Jun. 14, 2021.

(51) Int. Cl. C07K 14/78; C07K 1/14

(52) U.S. Cl. CPC C07K 14/78; C07K 1/145

(57) ABSTRACT

Processes are provided that yield high molecular weight collagen without enzymatic or chemical treatment, preserving triple helical content of native collagen. Processing conditions and energy input on animal tissue supply streams can be controlled to obtain collagen compositions with desirable yields and properties, including ratios of denatured and nondenatured collagen with reduced processing time and higher mass throughput.

### CROSS-REFERENCE TO RELATED APPLICATIONS

This application claims the benefit of U.S. Provisional Application No. 63/210,483, filed on Jun. 14, 2021, which is incorporated herein by reference in its entirety.

### FIELD OF THE INVENTION

The invention relates to methods for processing of animal tissue for production of collagen, in particular intact, triple helical, nondegraded, nondenatured collagen.

### BACKGROUND

The collagen protein, the most abundant protein in animals and much marine life, varies from species to species but all native collagen occurs in the form of a triple helix comprising three protein strands. The fiber-like structure of collagen is used to make connective tissue, and is a major component of bone, skin, muscles, tendons, and cartilage. Native collagens found in animal tissues are naturally crosslinked and thereby rendered largely insoluble in aqueous media, and therefore difficult to obtain and further exploit commercially in this form. Collagen has been widely extracted, purified and applied in diverse forms, foods, and technologies for centuries. More than 2000 years ago, early physicians used collagen to close wounds. Hydrolyzed extracted collagens are currently widely used in medical treatments including wound closure, treatment of burns, hemostasis, hernia repair, bone repair, cartilage repair, and dental applications. (Meyer, “Processing of collagen based biomaterials and the resulting material properties,” BioMed Eng OnLine, 18:24 (2019)).

Collagen extraction has traditionally been focused on obtaining gelatin, a denatured form of collagen protein, where mass extraction from animal hides raw materials sources utilized scaled, primarily chemical and biochemical processes not based on mechanical, hydrodynamic, or electrical extraction energies. Full-length nondenatured triple helix collagen has not been viewed as a desired product due to the difficulties extracting this native form of collagen from animal sources in suitable quantities and with attractive economies. Gelatin has historically been the commercial target using thermal and chemical extraction, and has been widely used as a nutritional source and matrix for diverse biomedical and cosmetic applications. Collagen extraction to yield gelatin has a long history based on batch-based, enzymatic (pepsin, trypsin, collagenase) and acid/base and thermal treatments that require considerable process time (days to weeks) in enzymatic or chemical vats to extract fragmented, denatured collagen and gelatin that are purified and marketed. The common alternative to enzymatic extraction of denatured collagen is the use of aqueous animal solids extractions at acidic pH (e.g., acetic acid solution at pH ˜3) that hydrolyze the crosslinking bonds between collagen strands to destroy the triple helix and fragment the polypeptide chains, or basic pH (e.g., sodium hydroxide solution at pH>8) that does the same. Thermal treatments (i.e., temperatures above 55° C.) are known to enhance chemical treatments for improved yields of denatured collagen from animal sources. Notably, none of these mass-yield, aqueous solution-based hydrolytic methods yield intact native triple helix collagen from animal sources. These processes destroy this hallmark native collagen protein structure, either the triple helix or the collagen length, or both, as a consequence of the processing, and the intent and motivation is to extract and produce hydrolyzed collagen fragments and gelatin protein strands, not intact native triple helical collagen.

Mechanical maceration of animal products to extract fat and proteins is known. Hydrodynamic shear is also known for pre-treatment of animal products to extract fat and protein. However, these known processes have not focused on extraction of native collagen, since enzymatic and chemical extraction is much more efficient, and yields hydrolyzed collagen and gelatin en masse at economic scales.

Generally, it is known that native protein structures can be disrupted during the course of extraction or isolation. “Structural perturbation of protein molecules occurs as a result of conditions of temperature, pH, and ionic strength as well as through the presence of denaturants (e.g., urea, guanidine), organic solvents, and surfactants. In addition, structural destabilization can result from exposure to hydrodynamic shear forces originating from shaking, sonication, mixing, vortexing, and flow through conduits.” (Bekard et al., “The effects of shear flow on protein structure and function,” Biopolymers, pp. 733-745 (2011)). To harvest native proteins retaining native conformations, lengths and functional structural features, such protein disruption during the extraction, isolation and purification process is therefore avoided with a deliberate strategy to preserve native protein features. This protein structural preservation process design is absent in collagen extraction where collagen native structure disruption is the basis for high efficiency collagen extraction in the form of hydrolyzed collagen fragments and denatured gelatin.

Conventionally, collagen has been harvested and isolated for commercial purposes from cows or pigs. Since these mammals may carry the prions responsible for spongiform encephalopathy (mad cow disease) and other transmissible pathogens, as well as for dietary, religious, and environmental reasons, there have been extensive efforts to obtain native triple helical (undenatured) collagen from other sources, such as fish. A recent review of these efforts is described by Salvatore, et al. in “Marine collagen and its derivatives: Versatile and sustainable bio-resources for healthcare,” Materials Science & Engineering C113 (2020). Generally, the process of isolating fish and marine collagen includes the steps of: separation and cleaning (2 hours); size reduction (1 hour); removal of non-collagenous components (6 hours to 3 days); collagen extraction treatment (2 to 6 days); and salt precipitation (2 days). From catfish, the collagen yield was reported as 16.8% for acid-solubilized collagen (ASC) and 28.0% for pepsin-soluble collagen (PSC), with no evidence for native, non-denatured native collagen harvest. Despite extensive efforts, the authors concluded that isolation of fish-derived collagen remains a challenge due in part to poor consistency in the results.

Singh, et al. described collagen extraction from fish in “Isolation and characterization of collagen extracted from the skin of striped catfish (Pangasianodon hypophtalmu),” Food Chemistry 124 (2011) 97-105. Initially, the fish skin was mixed with 0.1 M NaOH at 4° C. The pretreated skins were defatted with 10% butanol and the collagen precipitated and dialyzed to yield 5.1% of acid soluble collagen (ASC). The undissolved residue obtained after ASC extraction was soaked in 0.5M acetic acid with pepsin and stirred at 4° C. for 48 hours, subjected to precipitation and dialyzed to yield 7.7% of pepsin soluble collagen (PSC). Another method to extract collagen from fish was reported by Xu, et al. in “Purity and yield of collagen extracted from southern catfish (Silurus meridionalis Chen) skin through improved pretreatment methods,” Intl J Food Prop., pp. 5141-5153 (2017). A method was described in which the catfish skins were treated with sodium carbonate followed by defatting with isopropanol. Then the skins were soaked in aqueous hydrogen peroxide to remove color. The skins were washed with distilled water and soaked for two days in a solution of acetic acid and pepsin, and collagen was collected from the supernatant by centrifugation. No evidence for native non-denatured collagen product is shown.

One treatment for meat and fish processing is the application of a pulsed electric field (PEF). (See, e.g., Gomez et al., “Application of pulsed electric fields in meat and fish processing industries: An overview,” Food Research International 123 (2019) 95-105.) Although PEF application is a non-thermal technology, the resulting temperature increase associated with Joule heating from PEF must be considered with sensitive proteins. PEF equipment includes a pulse generator, treatment chamber, electrodes and system for control and data acquisition. Process parameters beyond temperature control that Gomez et al. suggest must be defined while designing a PEF process include the number of PEF pulses, pulse duration, pulse width, pulse shape, pulse specific and pulse frequency.

PEF processing has been reported as effective in defatting of a fish skin. Chotphruethipong, et al. (“Effect of Pulsed Electric Field-Assisted Process in Combination with Porcine Lipase on Defatting of Seabass Skin, J. Food Sci. pp 1799-1805 (2019)) reported that “PEF was conducted at various electric field strengths (16 kV/cm and 24 kV/cm) and number of pulses (n) including 360, 720, and 1080 pulses. Pulse width (τ) and pulse repetition time (t) were 0.10 and 20 ms, respectively. . . . The optimized condition for defatting was as follows: PEF with electric field strength of 24 kV/cm for 72 ms, PPL at 42.36 U/g dry matter, and hydrolysis time of 139.78 min.” No evidence for native non-denatured collagen is shown.

Chinese Patent Application CN10362776A1 reported a method for extracting collagen from anglerfish skin. The skins were treated with alkali and then defatted with butanol for 28 hours. The resulting skins were washed with water, pulverized, and heated to 55° C. in aqueous citric acid for six hours to produce a crude protein extract. This crude extract was then subjected to a pulsed electric field (PEF) processing at a frequency of 30 Hz and field strength of 25 kV/cm. No evidence for native non-denatured collagen is shown.

Although PEF has been described for pretreatment of animal products to extract fat, proteins, and other molecules, there has not been a focus on using PEF for native collagen extraction, since enzymatic and chemical extraction, which yields hydrolyzed collagen and gelatin, is much more efficient at yielding various forms of hydrolyzed collagen.

Combinations of chemical, enzymatic, thermal, and mechanical treatments of animal products to extract fats and proteins, or to facilitate their separation, are known. Again, these methods have not focused on native collagen extraction, since enzymatic and chemical extraction is much more efficient for production of gelatin products, and native collagen is destroyed as a result of this processing.

Despite these and many other efforts, there remains a need to extract nonhydrolyzed and non-denatured collagen from animal skins, especially fish skins, with higher process collagen yield, higher collagen product purity, and reduced collagen protein denaturing and fragmentation. Collagen denaturation can result from exposure to thermal energy (heat), exposure to acidic or basic solutions, exposure to detergents and surfactants, or exposure to mechanical energy (e.g., ultrasonic, mechanical or hydrodynamic shear). See, for example, Bekard, et al. (2011) Biopolymers 95(11):733-745: “Structural perturbation of protein molecules occurs as a results of conditions of temperature, pH, and ionic strength as well as through the presence of denaturants (e.g., urea, guanidine), organic solvents, and surfactants. In addition, structural destabilization can result from exposure to hydrodynamic shear forces originating from shaking, sonication, mixing, vortexing, and flow through conduits.”

High-throughput means to extract non-denatured native triple helical collagen by chemical or mechanical processes, or a combination thereof, have not been developed because the motivation to produce these processes has not been sufficient to balance the extraction difficulties and yields. Full length native trip helix collagens extracted from diverse animal sources are therefore difficult to extract in substantial quantities by any known process, and motivation to innovate such a process has been largely historically absent. Full length native triple helix collagens are currently of increasing and substantial commercial interest in several important applications, such as nutrition, cosmetics, medicine, and biotechnology, providing the new motivation to develop a method to extract full length collagens without denaturation from various animal sources at both mass scale and economy.

There is a need for new methods to efficiently extract collagen from animal tissues, particularly in full length nondenatured form. Methods that provide higher collagen product purity, improved native collagen quality by reduced collagen protein denaturing, reduced processing time for higher throughput, and reduced environmental impact would be desirable.

Collagen biochemical (e.g., acid, base, enzymatic) degradation and physical fragmentation (e.g., electrolysis, ultrasonic, mechanical, and hydrodynamic shear) from natural sources under current extraction conditions are common. Extraction of collagens by thermal, mechanical, physical, and chemical processing methods results in breakage of the triple helical bonds to form triple but non-helical (gamma), double (beta), and single (alpha) strands; these strands can also be fragmented into small collagen strand pieces. Intact triple helical fragments of various reduced sizes (short lengths, smaller molecular weights) also result from these same extraction processes. Full length, intact triple helical native collagen is not reported from current collagen mass extraction methods. Preservation of collagen native triple helical character chemistry, associated physical properties, and intact molecular weight of the native collagen molecule is therefore an opportunity and desired for numerous applications. Further, current methods for collagen extraction, even for denatured collagen, are arduous, complex, and require significant chemical treatment and processing times. More efficient, more economical, shorter duration and higher yielding processes for extracting all collagen products (i.e., denatured and native) from diverse animal raw material sources is desired.

### BRIEF SUMMARY OF THE INVENTION

Methods are provided for extracting collagen from isolated animal tissue. Compositions and applications of use that include the extracted collagen are also provided.

In one aspect, methods are provided for extracting collagen from isolated animal tissue, comprising: non-thermal mechanically and/or hydrodynamically shearing an animal tissue that comprises collagen in an aqueous media, thereby separating a collagen-containing liquid fraction comprising extracted collagen from a solid fraction, wherein the shearing is conducted under controlled conditions of temperature, pressure, and hydrodynamic energy such that at least about 5%, 10%, 15%, 20%, 25%, 30%, 35%, 40%, 45%, 50%, 55%, 60%, 65%, 70%, 75%, 80%, 85%, 90%, or 95% of the extracted collagen in the collagen-containing liquid fraction is in a nondenatured form. In some embodiments, the majority, e.g., greater than about 50%, 55%, 60%, 65%, 70%, 75%, 80%, 85%, 90%, or 95% of the extracted collagen in the collagen-containing liquid fraction is in the nondenatured form, e.g., the extracted nondenatured collagen has a molecular weight greater than about 220 kDa. In some embodiments, at least about 5%, 10%, 15%, 20%, 25%, 30%, 35%, 40%, 45%, 50%, 55%, 60%, 65%, 70%, 75%, 80%, 85%, or 90%, of the extracted collagen in the collagen-containing liquid is in a denatured or hydrolyzed form. In some embodiments, greater than about 50%, 55%, 60%, 65%, 70%, 75%, 80%, 85%, or 90% of the collagen in the collagen-containing liquid is in the denatured or hydrolyzed form.

In some embodiments, the collagen-containing liquid fraction includes at least about or greater than about 5%, 10%, 15%, 20%, 25%, 30%, 35%, 40%, 45%, 50%, 55%, 60%, 65%, 70%, 75%, 80%, 85%, 90%, or 95% of the collagen content in the isolated raw material animal tissue source from which it is extracted.

In some embodiments, the isolated animal tissue is maintained at a temperature that is less than about 10° C., less than about 7° C., or about 4° C. to about 7° C., prior to mechanical and/or hydrodynamic shearing. In some embodiments, microbial or enzymatic degradation of collagen or activation of endotoxins is limited in comparison to an isolated animal tissue raw material that is not maintained at a temperature less than about 10° C.

In some embodiments, the mechanical and/or hydrodynamic shearing of the isolated animal tissue that comprises collagen incudes one or more of: (i) low shear extrusion, optionally at about 100 sec⁻¹ or less, through an extruder that includes an inlet, a compression screw, a barrel, and a positive displacement pump, wherein the tissue is pumped through the barrel with the compression screw under pressure of about 10 psi to about 150 psi and with temperature conditions controlled such that the difference in temperature between the inlet and outlet does not vary more than about 20° C., wherein extruded material that flows through the outlet is further separated into the collagen-containing liquid fraction that comprises extracted collagen and the solid fraction; (ii) compression in a perforated compression cylinder, wherein the cylinder is compressed at a pressure of about 10 psi to about 150 psi and a cycle time of about 5 sec to about 30 sec, wherein the collagen-containing liquid fraction flows through perforations on the periphery of the cylinder; or (iii) processing through a dewatering press apparatus at a pressure of about 10 psi to about 150 psi and a residence de-watering time of about 5 sec to about 30 sec.

In some embodiments, the collagen-containing liquid fraction and/or the solid fraction is further processed under the same or different conditions in the same or different apparatus to improve yield and/or purity of the extracted collagen.

In some embodiments, prior to, concurrent with, or after shearing the isolated animal tissue, the tissue is subjected to an input of electric field and/or acoustic energy that does not substantially degrade the structure of the collagen extracted from the tissue. For example, the input energy may be provided by pulsed electric field and/or ultrasonication treatment. In some embodiments, the input of energy may include pulsed electric field treatment, for example, at an electric field strength that is about 3 kV/cm to about 90 kV/cm. In some embodiments, the input of energy may include ultrasonic treatment, for example, at about 20 Hz to about 40 Hz, about 20 Hz to about 30 Hz, about 25 Hz to about 35 Hz, or about 30 Hz to about 40 Hz, and about 150 W to about 250 W, about 150 W to about 200 W, about 175 W to about 225 W, or about 200 W to about 250 W instrument input power, and with processing time that varies depending on the sample and temperature. In some embodiments, the input of energy comprises about 40 kJ/kg to about 100 kJ/kg of the isolated animal tissue. Typically, the input of energy does not raise the temperature of the isolated animal tissue or a processing milieu in which it resides above the denaturation temperature of the collagen source, e.g., about 30° C., 35° C., or 40° C., depending on the species and tissue from which the collagen is derived.

In some embodiments, prior to mechanical and/or hydrodynamic shearing, optionally prior to an input of energy, the tissue, the isolated animal tissue is mechanically diced, macerated, minced or ground, thereby processing the tissue into smaller pieces under low shear conditions. For example, the isolated animal tissue may be mechanically diced or minced into pieces that have an average cross-sectional area of about 0.5 square inches to about 2 square inches, or about 1 square inch to about 2 square inches.

In some embodiments, the method does not include energy, chemical, and/or enzymatic conditions that intentionally denature or hydrolyze collagen, for example, conditions that intentionally degrade or hydrolyze collagen to produce fragments that are smaller than about 220 kDa.

In some embodiments, at least about or greater than about 70%, 75%, 80%, 85%, 90%, or 95% of the method is conducted at a temperature that is lower than the denaturation temperature for the collagen. In some embodiments, at least about 70%, 75%, 80%, 85%, 90%, or 95% of the method is conducted at a temperature that does not exceed 30° C. In some embodiments, at least about 70%, 75%, 80%, 85%, 90%, or 95% of the method is conducted at a mechanical shear rate that is below 100 sec⁻¹.

In some embodiments, the collagen-containing liquid fraction exhibits an aerobic plate count for microbial contamination less than about 104 CFU/g, 103 CFU/g, or 10² CFU/g. In some embodiments, the collagen-containing liquid fraction exhibits at least about 10²-fold, 10³-fold, or 10⁴-fold reduction in aerobic plate count for microbial contamination in comparison to the isolated animal tissue from which it was extracted.

In some embodiments, the isolated animal tissue is skin tissue. In one embodiment, the tissue is derived from marine or aquaculture waste, such as fish skin tissue.

In some embodiments, the collagen-containing liquid fraction is produced from the isolated animal tissue in less than about 12, 11, 10, 9, 8, 7, or 6 hours.

In some embodiments, a collagen-containing liquid, prepared by the methods described above, is provided, wherein at least about 5%, 10%, 15%, 20%, 25%, 30%, 35%, 40%, 45%, 50%, 55%, 60%, 65%, 70%, 75%, 80%, 90% or 95% of the extracted collagen is in the nondenatured form. Compositions that include isolated collagen that is extracted collagen purified from the collagen-containing liquid are also provided. For example, the extracted collagen has a weight greater than about 220 kDa. In some embodiments, at least about 5%, 10%, 15%, 20%, 25%, 30%, 35%, 40%, 45%, 50%, 55%, 60%, 65%, 70%, 75%, 80%, 90% or 95% of the collagen is in the nondenatured form.

In another aspect, a composition is provided that includes isolated collagen, wherein greater than about 50%, 55%, 60%, 65%, 70%, 75%, 80%, 85%, 90%, or 95% of the collagen is in a nondenatured form, for example, having a molecular weight greater than about 200 kDa.

In another aspect, a method is provided for extracting collagen from isolated animal tissue comprising: mechanically and/or hydrodynamically shearing an animal tissue that includes collagen in an aqueous media, thereby separating a collagen-containing liquid fraction comprising extracted collagen from a solid fraction, wherein the shearing is conducted under controlled conditions of temperature, pressure, and hydrodynamic energy such that at least about 5%, 10%, 15%, 20%, 25%, 30%, 35%, 40%, 45%, 50%, 55%, 60%, 65%, 70%, 75%, 80%, 85%, 90%, or 95% of the extracted collagen in the collagen-containing liquid fraction is in a denatured form. In some embodiments, the majority, e.g., greater than about 50%, 55%, 60%, 65%, 70%, 75%, 80%, 85%, 90%, or 95% of the extracted collagen in the collagen-containing liquid fraction is in the denatured form.

In some embodiments, mechanical and/or hydrodynamic shearing of the isolated animal tissue that includes collagen incudes one or more of: (i) low shear extrusion, optionally at about 100 sec⁻¹ or less, through an extruder that comprises an inlet, a compression screw, a barrel, and a positive displacement pump, wherein the tissue is pumped through the barrel with the compression screw under pressure of about 10 psi to about 150 psi, wherein extruded material that flows through the outlet is further separated into the collagen-containing liquid fraction that comprises extracted collagen and the solid fraction; (ii) compression in a perforated compression cylinder, wherein the cylinder is compressed at a pressure of about 10 psi to about 150 psi, and a cycle time of about 5 sec to about 30 sec, wherein the collagen-containing liquid fraction flows through perforations on the periphery of the cylinder; or (iii) processing through a dewatering press apparatus at a pressure of about 10 psi to about 150 psi, and a residence de-watering time of about 5 sec to about 30 sec. In some embodiments, the method is conducted without temperature controls, or at a temperature of about 40° C. to about 60° C.

In some embodiments, prior to, concurrent with, or after shearing the isolated animal tissue, the tissue is subjected to an input of electric field and/or acoustic energy, such as pulsed electric field and/or ultrasonication treatment. In some embodiments, the input of energy includes an electric field strength that is about 3 kV/cm to about 90 kV/cm. In some embodiment, the input of energy includes ultrasonic treatment at about 20 Hz to about 40 Hz, about 20 Hz to about 30 Hz, about 25 Hz to about 35 Hz, or about 30 Hz to about 40 Hz, and about 150 W to about 250 W, about 150 W to about 200 W, about 175 W to about 225 W, or about 200 W to about 250 W instrument input power. In some embodiments, the input of energy includes about 40 kJ/kg to about 100 kJ/kg of the isolated animal tissue.

In some embodiments, prior to mechanical and/or hydrodynamic shearing of the tissue, and optionally prior to input of electric field and/or acoustic energy, the isolated animal tissue is mechanically diced, macerated, minced or ground, thereby processing the tissue into smaller pieces under low shear conditions. For example, the isolated animal tissue may be mechanically diced or minced into pieces are an average cross-sectional area of about 0.5 square inches to about 2 square inches, or about 1 square inch to about 2 square inches.

In some embodiments, the isolated animal tissue is skin tissue, such as fish skin.

In some embodiments, the collagen-containing liquid fraction is produced from the isolated animal tissue in less than about 12, 11, 10, 9, 8, 7, or 6 hours.

In some embodiments, a collagen-containing liquid, prepared by the methods described above, is provided, wherein at least about 5%, 10%, 15%, 20%, 25%, 30%, 35%, 40%, 45%, 50%, 55%, 60%, 65%, 70%, 75%, 80%, 85%, 90%, or 95% of the extracted collagen is in the denatured form. In some embodiments, greater than about 50%, 55%, 60%, 65%, 70%, 75%, 80%, 85%, 90%, or 95% of the extracted collagen is in the denatured form. In some embodiments, the collagen is purified from the collagen-containing liquid.

In another aspect, compositions that include collagen (e.g., extracted, isolated, or purified collagen; nondenatured or denatured or a combination thereof) or a collagen-containing liquid, as described herein, may be included in A composition, such as, but not limited to a food, nutritional supplement, nutraceutical, animal feed, pharmaceutical dosing, drug delivery or gene carrier formulation, wound or burn care dressing, cosmetic additive in a gel, cream, salve, drop, ointment or topical dressing, cosmeceutical, collagen cell culture scaffold, cultivated meat or meat analogue, additive manufacturing 3-D printable matrix, 3-D bio-ink for tissue engineering, cell carrier, and/or medical device component, or container or packaging material.

### BRIEF DESCRIPTION OF THE DRAWINGS

FIG. 1 shows an example of a flow diagram for extraction of collagen from raw material in accordance with processes described herein.

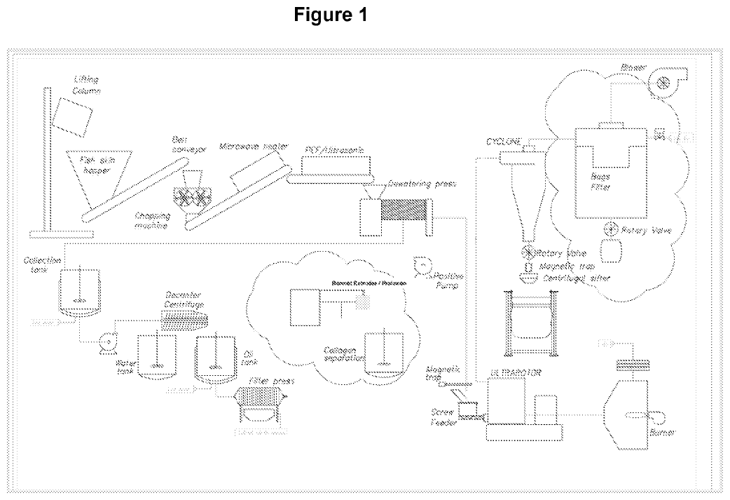

FIG. 2 shows an example of a flow diagram for pulsed electric field (PEF) pretreatment of animal tissue (e.g., skin tissue) as described herein.

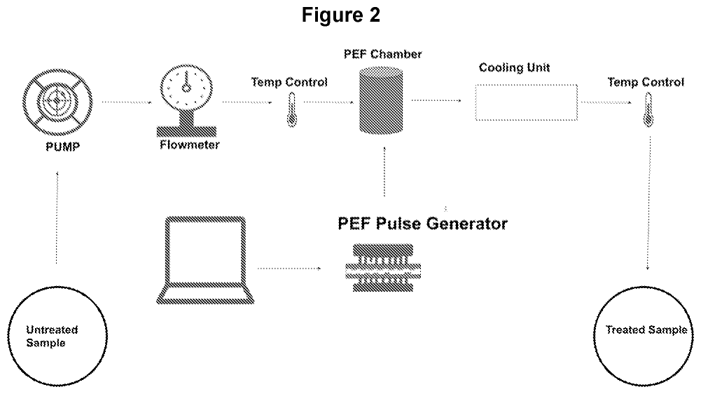

FIG. 3 shows an example of a circulation-type ultrasonic system.

FIG. 4 shows SDS-PAGE denaturing gel electrophoresis of collagenous extracts comparing PEF pre-treatments in both screw press and hydraulic press mechanical extractions. Lanes 1 and 2: hydraulic press only; Lanes 3 and 4 hydraulic press and PEF (40 kJ/kg); Lanes 5 and 6 screw extrusion only; Lanes 7 and 8 screw extrusion and PEF (96 kJ/kg); and Lane 9 gel standards using acid-soluble bovine commercial collagen. Gel separation size: 200 kDa-6.5 kDa.

### DETAILED DESCRIPTION

Provided herein are methods for collagen production. In some embodiments, the methods produce significant amounts of full length nondenatured collagen from available animal sources, such as, but not limited to marine sources, such as fish skin. In other embodiments, the methods produce denatured and/or hydrolyzed collagen from the same animal sources, but with altered process conditions, and notably greatly improved process time.

The methods described herein include combinations of mechanical, electrical, hydrodynamic, and/or thermal processing, which yield native, non-denatured collagen from a starting animal tissue at a higher mass throughput, greater extraction efficiencies (fractional yields), and in shorter processing times than methods that are currently used in the art. The disclosed methods may be used to produce commercially valuable quantities of collagen at mass scale under controlled combinations of extraction conditions, without chemical or thermal processing. In some embodiments, the methods described herein produce collagen of high nondenatured (e.g., nonhydrolyzed, triple helical) content, i.e., preserving higher molecular weight forms of collagen without hydrolysis and/or fragmentation, or substantially without hydrolysis and/or fragmentation, while preserving or substantially preserving triple helical content and native configuration and structure of collagen. In other embodiments, the non-chemical, non-proteolytic process conditions are altered to produce denatured and/or hydrolyzed collagen but at more rapid processing times than current methods. Processing times and non-thermal energy input conditions may be modulated to control fractions of both undenatured collagen product and denatured (gelatin) product output streams produced from animal tissue raw material supply input streams.

In certain embodiments, the collagen extraction methods described herein avoid the use of proteolytic enzymes, acid/base treatments, and/or thermal energy to increase yields of collagen or collagen constituents or derived elements or peptides. The standard of the gelatin and collagen extraction industries across all animal sources through the centuries is attributed to the key roles of thermal, enzymatic, and chemical adjustments in processing raw material as a means to enhance extraction.

Unexpectedly, particle size reduction and surface area expansion of the raw material with low or no shear extraction/pressing, optionally combined with pre- and/or post-processing step(s) exploiting pulsed electric field and/or acoustic energy treatment, enables the improved extraction of collagen from animal tissue raw materials, such as fish skin, with disproportionately higher levels of collagenous material. Advantages of the present invention include reduced processing time for extraction, lower capital and/or operating expenditures, and superior collagen product characteristics.

### Definitions

Unless defined otherwise herein, all technical and scientific terms used herein have the same meaning as commonly understood by one of ordinary skill in the art to which this invention belongs. Singleton, et al., Dictionary of Microbiology and Molecular Biology, second ed., John Wiley and Sons, New York (1994), and Hale & Markham, The Harper Collins Dictionary of Biology, Harper Perennial, NY (1991) provide one of skill with a general dictionary of many of the terms used in this invention. Any methods and materials similar or equivalent to those described herein can be used in the practice or testing of the present invention.

Numeric ranges provided herein are inclusive of the numbers defining the range.

“A,” “an” and “the” include plural references unless the context clearly dictates otherwise.

The term “about” as used herein when referring to a measurable value such as an amount, a temporal duration, and the like, is meant to encompass variations of ±5%, 1%, or ±0.1% from the specified value, as such variations are appropriate to perform the disclosed methods or in connection with a disclosed composition.

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

“Aqueous media” refers to a liquid milieu in which the majority component or phase is water, but which may also include dissolved or added or extracted salts, proteins, fats, and/or lipids (e.g., from the raw material isolated animal tissue), and/or co-solvent molecules, and may also contain insoluble suspended solids, in various proportions as mixtures.

In the claims, as well as in the specification, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively.

“Denatured” when used in reference to collagen herein, refers to the loss of its native triple helical (i.e., type II left-handed helix) structure, typically by triple helix chemical, thermal, enzymatic, or mechanical degradation, or separation of the three helical strands to eliminate helical content, or hydrolysis to produce smaller polypeptides. (See, e.g., Ramshaw, et al. (2014) Bioengineered 5(4): 227-233) Denatured collagen may be in the form of gelatin, which typically consists of single collagen-derived chains or strands no longer associated or folded into the native triple-helical structure.

“Nondenatured” collagen refers to substantially full length, intact (e.g., triple helical) collagen, i.e., native configuration, and substantially nondegraded (e.g., not hydrolyzed to smaller length fragments and not dissociated into single strands).

The “denaturation temperature” of collagen refers to the equilibrium temperature producing measurable loss of native triple helical structure by locally dissociating strands within the collagen molecule.

The term “derived from” or “extracted from” encompasses the terms “originated from,” “obtained from,” “obtainable from,” “isolated from,” and “created from,” and generally indicates that one specified material finds its origin in another specified material or has features that can be described with reference to another specified material. “Isolated” indicates that a substance is separated from a material or environment with which it is associated in nature. For example, “isolated collagen” may refer to collagen that has been extracted from a tissue (for example, skin tissue) with which it is associated in a natural, living system. Additionally, “strands” may be isolated from triple helical collagen by energy that pulls strands apart into complete separation.

“Hydrolyzed” or “hydrolysis” refers to the chemical breakdown of a compound (e.g., collagen) due to reaction with water. Hydrolysis of collagen alters the collagen molecular chemistry, e.g., specifically breaking chemical bonds or substituting chemical associations in the native molecule.

The term “lipid” herein refers to one or more water-insoluble molecules (e.g., biomolecules) that include a fatty acyl group (e.g., saturated or unsaturated acyl chains). For example, the term “lipids” includes oils, phospholipids, free fatty acids, monoglycerides, diglycerides, and triglycerides.

“Non-thermal” refers to energy sources that by themselves are not intended to produce heat through their application; where any thermal changes as a result of their application are consequence of conversion of the intended energy input into heat as a secondary energetic process. For example, PEF and acoustic energy are defined as non-thermal processes.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein, “polypeptide” refers to a composition comprised of chemically linked amino acids and recognized as a protein by those of skill in the art. The conventional one-letter or three-letter code for amino acid residues is used herein. The terms “polypeptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also, included within the definition are, for example, polypeptides containing one or more analogs of an amino acid (including, for example, unnatural amino acids, enzymatically modified amino acids, etc.), as well as other modifications known in the art.

The terms “recovered,” “isolated,” “purified,” and “separated” as used herein refer to a material (e.g., a protein) that is removed from at least one component with which it is naturally associated. For example, these terms may refer to a material which is substantially or essentially free from components which normally accompany it as found in its native state, such as, for example, an intact biological system. “Isolated animal tissue” refers to a raw material animal tissue source, such as skin tissue, that is no longer part of an intact living biological system, and that is used as a starting material for collagen extraction in a method as described herein.

### Collagen Source Material

Any type of animal tissue that contains collagen may be used as a starting material in the collagen production processes described herein. In some embodiments, the animal tissue is skin tissue, such as, but not limited to, fish skin, poultry (e.g., chicken) skin, pork skin, beef skin, etc. In some embodiments, a marine collagen tissue source, or aquaculture tissue source, is used, such as fish skin. In certain embodiments, the source is a Siluriformes species (e.g., a catfish species), such as catfish skin. In one embodiment, the tissue source is skin of a Pangasius species.

The processes described herein may include feeding relatively large pieces of the collagen tissue source (e.g., skin tissue, for example, fish skin) (for example, at least 90 mass % of the feed in pieces having a length of at least about 10 cm, or at least about 20 cm, or in the range of about 20 cm to about 80 cm). Alternatively, the tissue piece length can be in the range of about 2.5 cm to about 10 cm or smaller. In some embodiments, a first operation involves size reduction of the tissue (e.g., skins) from their natural length in excess of 10 inches or more; this can be accomplished through standard meat processing systems including but limited to grinding, dicing, mincing, slicing, etc. In some embodiments, the ideal size for processing is approximately 1-3 inches in length and/or width or about 1 to about 2 square inches in cross sectional area. In some embodiments, the tissue source (e.g., animal skin, such as fish skin), is subjected to mechanical size reduction under low shear conditions.

Typically, the tissue source (e.g., animal skin, such as fish skin) is chilled or frozen after harvesting (e.g., immediately after harvesting, ideally, material should be chilled immediately after harvesting), and then maintained at a low temperature below the collagen denaturation temperature prior to collagen extraction. This may have the effect of minimizing collagen denaturation, microbial growth, enzymatic or microbial breakdown of collagen, and/or release and/or activation of microbial endotoxins. For example, the tissue may be frozen after harvesting and stored frozen (e.g., minus 20° C. to minus 70° C., or the harvested tissue may be processed immediately at a temperature not to exceed 10° C. or 7° C. For example, the tissue may be maintained after harvesting at a temperature that is about 10° C. or lower, or about 4° C. to about 7° C., such as at about 4° C.

### Pulsed Electric Field or Acoustic Energy Pretreatment

In some embodiments, the collagen tissue source (optionally subjected to size reduction, as described above) may be pretreated and/or post-treated with an input of energy density, such as pulsed electric field and/or acoustic (e.g., ultrasonication) energy under controlled temperature, preferably below the collagen denaturation temperature (e.g., below 30° C.). Energy input is controlled at a level insufficient to degrade or that does not substantially degrade the native collagen structure (e.g., triple helical structure) in the tissue. Although not wishing to be bound by theory, the energy pretreatment is intended to disrupt inter-molecular bonds that bind collagen to tissue (e.g., skin epidermal) material, for example, non-covalent dipole bonds binding collagen fibrils in an epidermal matrix), which weakens this interaction to facilitate downstream extraction of the collagen.

In some embodiments, the energy pretreatment does not increase the temperature of the process media or tissue source above the denaturation temperature of collagen. For example, for fish skin as a collagen source, the temperature of the tissue or processing milieu in which it sits may be maintained at about 30° C. or lower, about 25° C. or lower, about 20° C. to about 30° C., about 20° C. to about 25° C. or about 26° C., about 25° C. to about 30° C., or about 25° C. to about 26° C. In some embodiments, the temperature of the tissue or processing milieu in which it sits may be maintained at a temperature that is about 40° C. or lower, about 30° C. or lower, or about 20° C. or lower, or any of about 20° C. to about 30° C., about 25° C. to about 30° C., about 25° C. to about 35° C., or about 30° C. to about 40° C.

In some embodiments, the energy pretreatment includes energy input of about 40 kJ/kg to about 100 kJ/kg of tissue or any of about 40 kJ/kg or about 60 kJ/kg, about 50 kJ/kg to about 70 kJ/kg, about 60 kJ/kg to about 80 kJ/kg, about 70 kJ/kg to about 90 kJ/kg, or about 80 kJ/kg to about 100 kJ/kg, or any of about or at least about 40 kJ/kg, 45 kJ/kg, 50 kJ/kg, 55 kJ/kg, 60 kJ/kg, 65 kJ/kg, 70 kJ/kg, 75 kJ/kg, 80 kJ/kg, 85 kJ/kg, 90 kJ/kg, 95 kJ/kg, or 100 kJ/kg.

### Pulsed Electric Field (PEF)

Depending on product stream preferred (i.e., native collagen versus denatured collagen and gelatin), the raw feed materials can be thawed, then mechanically or, for gelatin product, chemically, enzymatically, and/or thermally pretreated, and then treated with PEF, or treated with PEF without selected pretreatments, or treated with PEF both before and after selected pretreatments. Preferably, for native, non-denatured collagen product, the thawed raw material is diluted with at least about 20 mass % aqueous media, preferably about 30 to about 70 mass % aqueous media, which keeps processing temperatures low (e.g., below about 70° C., preferably about 50° C. or less, more preferably about 40° C. or less, more preferably about 30° C. or less than about 30° C., and in some embodiments between about 1° C. and about 20° C., about 4° C. to about 25° C. or about 26° C., about 25° C. or lower or about 26° C. or lower, or about 25° C. to about 30° C.), and reduces or prevents collagen denaturation while providing energy input to facilitate collagen extraction from raw feed materials.

Surprisingly, in the absence of chemical, thermal, and enzymatic pretreatment, PEF treatment of animal tissue, such as fish skins, followed by mechanical and/or hydrodynamic shearing and collagen extraction, such as pressing, significantly increased the relative proportion of collagen extracted, in comparison to an identical process that does not include PEF pretreatment. In our testing with fish skin samples, we found that pressing (e.g., extrusion or other compression-based collagen extraction procedure as described herein) alone at room temperature yielded an extract with no detectable collagen, while the sample fish skins subjected to a PEF pretreatment and then pressed at room temperature yielded an extract with 1.68% collagen by mass. In preferred embodiments, collagen is recovered from an animal tissue source as described herein without the use of collagen-degrading or denaturing enzymes, such as proteolytic enzymes, and without acid or base pretreatment of the tissue, e.g., fish skins. In some preferred embodiments, PEF is combined with other mechanical and hydrodynamic shear forces to separate collagen.

Preferably, the applied PEF power and treatment duration is sufficient to kill microorganisms in the feed but less than the energy at which collagen is either denatured or fragmented. Field strength, temperature, pulse width, numbers of pulses and frequency of pulses are all important to achieving product extraction from raw materials (i.e., animal tissue, such as skin tissue, e.g., fish skin) while also killing resident microbes (i.e., reducing microbial colony forming units (CFUs)).

Conditions for PEF use a variety of parameters including field strength, electrode distance, frequency, pulse width, energy per pulse, and applied energy per mass, involving all of these plus the applied PEF time (duration). In some preferred embodiments, parameters may include one or any combination of the following: field strength of at least about 1 kV/cm or at least about 2 kV/cm or in the range of about 1 to 10 kV/cm, or about 3 kV/cm to about 90 kV/cm, an electrode distance of between about 3 and about 20 cm or between about 5 and about 15 cm; a pulse frequency or at least about 10 Hz or in the range of about 5 to about 50 Hz, or about 10 to about 30 Hz; a pulse width of at least about 0.2 μsec, or at least about 3 μsec or in the range of about 0.2 μsec to about 2000 μsec, about 3 to about 15 μsec, or about 3 to about 10 μsec; an energy of at least about 20J or at least about 40 J or in the range of about 30 to about 150 J per pulse; a total applied energy of at least about 50 kJ/kg or at least about 70 kJ/kg or in the range of about 60 to about 150 kJ/kg or about 70 to about 120 kJ/kg.

The proportions of collagen to total protein in the extract from a press is preferably at least about 10% or at least about 20% or at least about 25%, or in the range of about 10% to about 70% or about 10% to 50%. In view of the descriptions herein, the worker of ordinary skill can adjust the parameters to obtain these proportions through no more than routine experimentation.

In some embodiments, PEF (and optionally the entire collagen extraction process) is performed at temperatures of about 40° C. or less, about 35° C. or less, about 30° C. or less, about 25° C. or less, about 20° C. or less, 0° C. to about 25° C., about 20° C. to about 30° C., about 30° C. to about 40° C., about 25° C. or about 35° C., about 20° C. to about 25° C. or about 26° C., or about 4° C. to about 25° C. or about 26° C. PEF processing may result in some feed stream heating, depending on field strength, pulse width, and numbers of pulses, but control of energy duration and intensity can facilitate control of feed temperature excursions and reduces or eliminates collagen triple helical denaturation and collagen fragmentation.

In some embodiments, PEF is conducted at electric field strength about 3 kV/cm to about 90 kV/cm, about 3 kV/cm to about 10 kV/cm, about 5 kV/cm to about 15 kV/cm, about 10 kV/cm to about 50 kV/cm, about 20 kV/cm to about 90 kV/cm, about 5 kV/cm to about 50 kV/cm, about 15 kV/cm to about 75 kV/cm, about 20 kV/cm to about 60 kV/cm, about 5 kV/cm to about 25 kV/cm, about 50 kV/cm to about 80 kV/cm, or about 60 kV/cm to about 90 kV/cm.

In some embodiments, PEF is conducted at pulse width about 0.2 ps (microseconds) to about 2000 μs, about 0.2 μs to about 100 μs, about 1 μs to about 500 μs, about 1 μs to about 10 μs, about 10 μs to about 100 μs, about 1 μs to about 50 μs, about 0.2 μs to about 2 μs, about 0.2 μs to about 0.5 μs, about 0.2 μs to about 5 μs, about 0.2 μs to about 10 μs, about 500 μs to about 1000 μs, about 1000 μs to about 1500 μs, about 1500 μs to about 2000 μs about 1000 μs to about 2000 μs m about 0.2 μs to about 0.5 μs, about 1 μs to about 5 μs, about 10 μs to about 50 μs, about 100 μs to about 150 μs, about 200 μs to about 250 μs, about 300 μs to about 350 μs, about 400 μs to about 450 μs, about 500 μs to about 550 μs, about 600 μs to about 650 μs, about 650 μs to about 700 μs, about 700 μs to about 750 μs, about 750 μs to about 800 μs, about 800 μs to about 850 μs, about 850 μs to about 900 μs, about 900 μs to about 950 μs, about 950 μs to about 1000 μs, about 1000 μs to about 1100 μs, about 1100 μs to about 1200 μs, about 1200 μs to about 1300 μs, about 1300 μs to about 1400 μs, about 1400 μs to about 1500 μs, about 1500 μs to about 1600 μs, about 1600 μs to about 1700 μs, about 1700 μs to about 1800 μs, about 1800 μs to about 1900 μs, or about 1900 μs to about 2000 μs,

In some embodiments, PEF is conducted at number of pulses about 50 to about 300, about 50 to about 100, about 100 to about 150, about 150 to about 200, about 200 to about 250, about 250 to about 300, about 50 to about 150, about 100 to about 300, about 50 to about 200, or about 200 to about 300.

In one embodiment, PEF is conducted at temperature about 30° C., electric field strength about 25 kV/cm, pulse width about 2 s, number of pulses about 300, and frequency about 20 to about 50 Hz, about 50 Hz to about 100 Hz, about 250 to about 500 Hz, about 250 Hz to about 1000 Hz, about 20 Hz to about 1800 Hz, about 500 to about 1000 Hz, about 800 Hz to about 1500 Hz, about 1000 Hz to about 1800 Hz, or any of about 10 Hz, 20 Hz, 50 Hz, 100 Hz, 150 Hz, 200 Hz, 250 Hz, 300 Hz, 350 Hz, 400 Hz, 450 Hz, 500 Hz, 550 Hz, 600 Hz, 650 Hz, 700 Hz, 750 Hz, 800 Hz, 850 Hz, 900 Hz, 950 Hz 1000 Hz, 1050 Hz, 1100 Hz, 1150 Hz, 1200 Hz, 1250 Hz, 1300 Hz, 1350 Hz, 1400 Hz, 1450 Hz, 1500 Hz, 1550 Hz, 1600 Hz, 1650 Hz, 1700 Hz, 1750 Hz, or 1800 Hz.

In another embodiment, PEF is conducted at temperature about 30° C., electric field strength about 60 kV/cm, pulse width about 0.2 μs, number of pulses about 50, and frequency about 10 Hz. All of these PEF energy conditions can be modified independently to produce different PEF applications, energy inputs and extraction results. This versatility is especially useful to adapt to different raw materials of different animal species, sizes, densities, collagen types, collagen fibrillar arrangements in tissue sources, and desired product streams (native triple helical non-denatured collagen versus denatured collagen and gelatin, or combinations thereof).

### Ultrasonic (Acoustic) Energy

Samples can be pretreated with ultrasound (acoustic energy) or treated during or after pressing (i.e., pretreatment, concurrent treatment, and/or posttreatment). In some embodiments, acoustic energy may be applied in conjunction with or as an alternative to PEF. Conditions and apparatus for apparatus are well known and can be selected through routine experimentation. Process temperature control can allow selection of triple helical collagen product (i.e., at processing temperatures below the collagen denaturation temperature) or denatured collagen and gelatin (at processing temperatures above the collagen denaturation temperature). Although not wishing to be bound by theory, the application of ultrasonic energy induces acoustic cavitation that disrupts the linkages binding the collagen within the tissue, e.g., subdermal layers of skin. This can result in sonoporation, de-texturization, and fragmentation of the skin cellular structure, and also local heating; thus, it may be desirable to limit application of acoustic energy so as not to heat or degrade the native collagen structure.

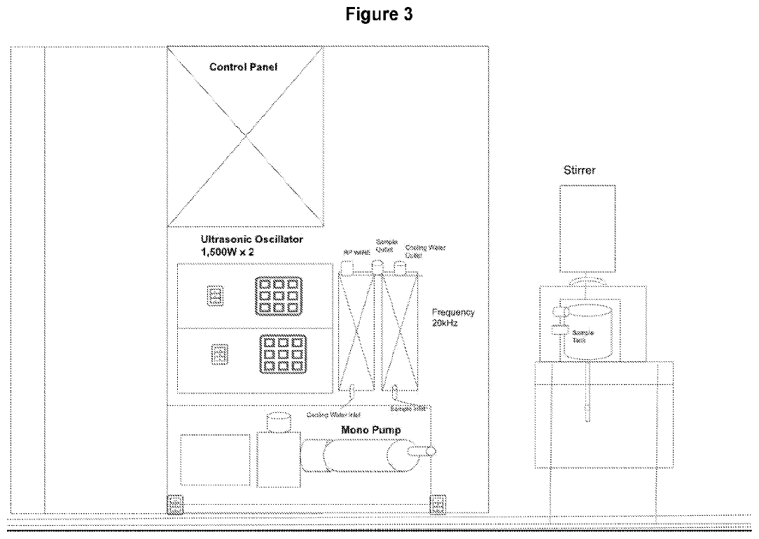

Acoustic energy supplied by an ultrasonic energy generator can be applied to animal raw materials (e.g., skin tissue, such as fish skin) to pretreat and/or post-treat collagen sources. Ultrasonic processing energy input conditions that result in a 50% or greater increase in collagen extraction yield over conventional collagen extraction (i.e., without aid of ultrasonic energy) are typical. A circulation-type ultrasonic system of total volume of 8 liters (e.g., from Mirae Ultrasound Co., Ltd.) is capable of achieving this. This scalable system includes a sample inflow port, conduit or channel, ultrasonic energy operator chamber, ultrasonic energy generator chamber, ultrasonic energy control unit, a recirculating pump, ultrasonic oscillator, a pump, and process cooling unit. A nonlimiting embodiment of such a system is shown in FIG. 3.

### Oil, Lipid, Fat, Fatty Acid and Triglyceride, and Microbial Endotoxin, Extraction

In some embodiments, supercritical fluid CO₂ extraction or another solvent extraction method is deployed to extract and separate lipids from proteins (e.g., collagen), either as a pretreatment of the starting animal tissue source (e.g., skin tissue, such as, but not limited to, fish skin) and/or as a posttreatment of the collagen-containing liquid product stream. In some embodiments, the supercritical fluid CO₂ extraction or solvent extraction removes or reduces content of endotoxins in the starting animal tissue feed and/or in the collagen-containing product. The supercritical fluid CO₂ extraction or solvent extraction is performed at a temperature that does not cause denaturation or degradation of the collagen. In some embodiments, supercritical fluid CO₂ extraction or solvent extraction reduces or avoids saponification in the starting animal tissue feed and/or in the collagen-containing product stream. Nonlimiting examples of conditions for supercritical fluid extraction may be found in: Ibanez, et al. (2016) Supercritical Fluid Extraction. Encycl. Food Health 227-233; Sousa, et al. (2020) J Polym Res 27:73; and Jamalludin, et al. (2022) Bioresour. Bioprocess 9:21, which are incorporated by reference herein in their entireties.

Examples of extractants for non-water soluble products of collagen extraction include supercritical CO₂, or supercritical CO₂ mixed with minority phases of water, or supercritical CO₂ mixed with minority phases of ethanol, or supercritical CO₂ mixed with minority phases of water and a GRAS (FDA-designated Generally Regarded As Safe) additive capable of forming micellar phases in supercritical fluids. Specifically, desired defatting and lipid removal pre-and/or post-treatment using supercritical fluid extraction (SFE) to extract collagen from animal sourced raw materials is achieved using SFE. This method is scalable to industrial mass throughput, practically sterile, certified as GRAS for food and cosmetic ingredient purification and operational at temperatures suitable to avoid collagen denaturation. In some embodiments, collagen feed sources either before or after size reduction and other processing described herein, are dewatered (e.g., using a Vincent dewatering press) and dispersed as small solid fragments (e.g., mechanical grinding or mincing to fragments less than 1 cm in any dimension) in the supercritical CO₂ majority milieu and subject to SFE.

Bacterial endotoxin removal from extracted collagen solid pieces may also be facilitated using these same or similar SFE conditions. (See, e.g., J. Supercrit Fluids (2011) 55(3):1052-1058; J. Supercrit Fluids (2008) 45(1):51-56; and U.S. Pat. No. 9,296,981B2, which are incorporated by reference herein in their entireties.)

### Pressure-Based Collagen Extraction

All or a portion of an animal tissue sample (e.g., skin tissue, such as fish skin tissue), optionally after size reduction and/or energy input (e.g., PEF and/or acoustic energy treatment), can be mechanically and/or hydrodynamically processed (e.g., mechanically and/or hydrodynamically sheared) under pressure (i.e., pressed), thereby yielding a collagen-containing liquid fraction, which may be separated from a residual solid fraction. For example, the raw material tissue (optionally pretreated as described above using any or all processes described herein) may pressed through orifices such as a screen, e.g., orifices having maximum openings of about 2 to about 15 mm or about 2 to about 5 mm. In some embodiments, optionally following suitable pretreatment, simple mechanical pressing yields desired collagen extraction results. Advantageously, pressing (and optionally the entire collagen extraction process) can be performed at a temperature that is lower than the denaturation temperature for collagen, which may vary depending on the starting tissue source and/or species from which the collagen is derived. For example, in some embodiments, for fish skin (such as, but not limited to skin of a Siluriformes species, e.g., skin of a Pangasius species), pressing (and optionally the entire collagen extraction process) can be performed at temperatures of about 40° C. or less, preferably about 35° C. or less, or about 30° C. or less, or about 26° C. or less, or about 25° C. or less, or about 20° C. or less, or 0° C. to about 25° C., or about 4° C. to about 25° C. or about 26° C. or about 30° C.

In some embodiments, pressing yields collagen in the collagen-containing liquid fraction that is any of at least about 5%, 10%, 15%, 20%, 25%, 30%, 35%, 40%, 45%, 50%, 55%, 60%, 65%, 70%, 75%, 80%, 85%, 90%, or 95% in a nondenatured form, i.e., molecular weight greater than 220 kDa, e.g., triple helical collagen. In some embodiments, greater than any of about 50%, 55%, 60%, 65%, 70%, 75%, 80%, 85%, 90%, or 95% of the collagen is nondenatured. In some embodiments, conditions may be deployed such that a substantial portion of the extracted collagen is in a denatured form, i.e., any of about 20%, 25%, 30%, 35%, 40%, 45%, 50%, 55%, 60%, 65%, 70%, 75%, 80%, 85%, 90%, or 95% is denatured (e.g., gelatin). In some embodiments, greater than any of about any of about 50%, 55%, 60%, 65%, 70%, 75%, 80%, 85%, 90%, or 95% of the collagen is denatured. In some embodiments, collagen may be extracted in combinations of denatured and nondenatured forms at desired ratios, by adjusting the processing and pressing conditions accordingly.

In some embodiments, a screw process is utilized, wherein the animal tissue material is compressed against a screen to facilitate the expression of the loosely bound collagen fibers. The desired shear can be obtained using a twin screw where the compression is created by conveying the tissue (e.g., skin tissue) and compressing it against the enclosing mesh screens. A nonlimiting example of an apparatus for such as process can be obtained from Vincent Corp. Alternatively, a single screw with a progressive flight orientation to provide conveying and pressure may be utilized. The parameters of material viscosity, screw profile and rotational speed can be adjusted. For example, a variable pitch screw can be utilized to increase compression, resulting in higher shear forces sufficient to produce the improved separation of collagen from raw material animal tissue. Increased screw speed will also increase processing shear rate and extraction yield.

In other embodiments, a dewatering press is utilized, such as a Vincent dewatering press. In one embodiment, the press includes a single screw inside a slotted sleeve wherein the raw material tissue (e.g., skin) is conveyed along the screw being exposed to mild shear and mild compression. The collagenous materials are forced through a sieved liner with small openings to facilitate the extraction of more free-flowing liquids. In this configuration, separation may be only partially complete as the single screw may not have sufficient functional capability to convey an extremely slippery tissue source, such as skin. In another embodiment, a two-screw configuration is utilized, which has increased conveying capacity and therefore may be capable of increased shear and compression. Thus, in some embodiments, extrusion is preferably conducted using a two-screw extruder.

In other embodiments, collagen is extracted through hydraulic compression in an enclosed cylinder. The pressure can be created using a hydraulic ram where pressure can be applied at levels in excess of about 100 psi. In this instance there is minimal shear, and the extraction of the collagen occurs without mechanical shear. This can be an adiabatic process, where the energy is applied through mechanical compression. The process cycle time and pressure application are controlled to minimize temperature increases that can negatively affect the quality of the protein extracted.

In another embodiment, an apparatus such as Protecon Meat Recovery system can be utilized. This system operates on the principle of differential extraction using a piston in a cylinder to compress the tissue (e.g., skin tissue) during which liquid collagen flows through perforations in the cylinder wall. In differentiation from normal operation where meat is separated from bones under high pressure, in this application durable skin is substituted in place of bone, liquid collagen is separated rather than meat proteins. Compared to separating meat from bone, in order to achieve separation of collagen from tissue (e.g., skin), operation is at lower piston speeds and reduced pressures. Tissue (e.g., skin) and collagen subjected to high pressure behave as incompressible fluids with differential flow properties related to their differential visco-elastic properties. The collagen can be separated as it flows through concentric slotted rings at the bottom of the compression cylinder.

In all processes described herein, it is desirable to minimize the use of chemical additives (e.g., acids or bases that alter media pH substantially from neutral), and processes that use excessive amounts of water. This results in a much more sustainable and environmentally desirable outcome, and improved triple helical content for the collagen product.

In some embodiments of the methods described herein, collagen may be extracted from the animal tissue starting raw material (e.g., skin tissue, such as fish skin) in 6 hours or less. In some embodiments, collagen is extracted from an animal tissue raw material source in any of 12, 11, 10, 9, 8, 7, or 6 hours or less. Different starting raw materials required different conditions and/or longer processing times. Depending on the raw material source, the number and types of pre- and/or posttreatment steps may also vary for effective collagen extraction.

As discussed above, conditions may be modulated to produce substantially nondenatured or denatured collagen or desired ratios of combinations thereof. In some embodiments, the collagen that is extracted (e.g., in 12, 11, 10, 9, 8, 7, or 6 hours or less) is substantially in a nondenatured form (e.g., molecular weight greater than about 220 kDa), e.g., at least about 20%, 25%, 30%, 35%, 40%, 45%, 50%, 55%, 60%, 65%, 70%, 75%, 80%, 85%, 90%, or 95% is nondenatured. In some embodiments, greater than any of about 50%, 55%, 60%, 65%, 70%, 75%, 80%, 85%, 90%, or 95% of the collagen is nondenatured. In some embodiments, the collagen that is extracted (e.g., in 12, 11, 10, 9, 8, 7, or 6 hours or less) is any of at least about 20%, 25%, 30%, 35%, 40%, or 45% nondenatured. In some embodiments, greater than any of about 50%, 55%, 60%, 65%, 70%, 75%, 80%, 85%, 90%, or 95% denatured.

In some embodiments, two or more pressing processes and/or apparatus are deployed sequentially to improve the yield of extracted collagen. For example, the tissue sample (e.g., skin tissue) may be pressed to yield a collagen-containing liquid fraction, and then the collagen-containing liquid fraction pressed again (under different conditions in the same apparatus or in a different apparatus) to yield greater purity or quality of extracted collagen product.

### Optional Additives

Prior to pressing or in conjunction with pressing, the animal tissue feed raw material can be treated with a soluble salt or salts, such as sodium chloride or potassium chloride, additionally phosphates such as sodium tripolyphosphate can be used to increase the solubility of proteins, non-proteolytic enzymes (e.g., non-collagen degrading enzymes such as lipases, elastases, esterases, hydrolases, lysozyme), antimicrobial peptides (nicin), and/or acids or bases, which may aid with release of protein to enhance product yield and quality, reduce or eliminate microbial contamination, and/or reduce other steps and time in processing while eliminating or substantially reducing undesired proteolytic degradation of collagen. In some embodiments, acidic or alkaline conditions can improve recovery and overall yields of hydrolyzed collagen. In some embodiments, the materials stream flowing through the apparatus, such as an extruder, can be acidified, for example acidified to a pH range of about 3 to about 5, or about 3.5 to about 4.5, with a food-grade acid, such as citric acid. Alternatively, alkaline conditions, for example pH about 8 and about 9, can be used. Salts can be added, in some embodiments, not to exceed 4 mass %; for example, about 2.5% to about 4% (w/w).

In some embodiments, a select enzyme or select enzyme combinations can be added, and non-proteolytic enzymatic extraction can be conducted during the pressing (e.g., extrusion) process to enhance collagen extraction. In some embodiments, one or more of a lipase, esterase, elastase, or hydrolase is included that does not degrade collagen, or under conditions in which collagen is not substantially degraded. In some embodiments, pepsin, elastase, lysozyme, or similar proteolytic enzymes can be added to facilitate release of collagen but under select conditions that limit or substantially avoid collagen degradation and fragmentation. In some embodiments, one or more antimicrobial agent or peptide, such as nisin, is included. Microbially induced enzymatic degradation of collagen is a natural undesired consequence of non-sterile food harvest and the microbes also produce proteases that damage collagen. Addition of the enzyme(s) and/or antimicrobial peptide(s) described herein may reduce or eliminate microbial contamination and/or growth in the extracted collagen-containing liquid fraction.

In some embodiments, one or more lipases and/or esterases may be added to break down endogenous lipids and fats contained in animal tissue raw materials.

### Extrusion

In some embodiments, a collagen-containing liquid fraction is extracted from an animal tissue source raw material, such as skin tissue, e.g., fish, poultry, pork, beef, etc. skin, via extrusion. A nonlimiting example of an extrusion system which may be adapted for use in the methods described herein is disclosed in U.S. Pat. No. 8,628,815, which is incorporated by reference herein in its entirety.

Pumps used in the extrusion process described herein are preferably positive displacement pumps. Commercial pumps are available from manufacturers such as Moyno, Waukesha, Vemag, or Marlen. Pressure on the tissue stream in the extruder is typically in the range of about 10 psi to about 100 psi, or about 10 psi to about 50 psi, through the extruder. In some embodiments, the extruder contains an auger screw that has a constant pitch. The extruder can have smooth walls in some embodiments, or in other embodiments, a rifled barrel. The interior extruder walls are typically cylindrical and the screw has an increasing root diameter. The outlet is typically a ring-shaped open annulus. In other embodiments, the outlet may have a restrictor which can be used to achieve a pressure and obtain some separation. In further embodiments, the interior flow path through the extruder has a frustopyramidal or frustoconical shape.

Preferably, extrusion is performed using a combination of shear which is created by the movement of the screw in relation to the barrel wall, controlled heating, in which the barrel wall and the hollow flight screw are independently controlled, and pressure that is controlled by a positive displacement feed pump and restricted flow through the exit of the extruder.

Typically, the extrusion is conducted in a single extruder; however, the process may be conducted in several extruders, for example, staged in four sequential extruders. An advantage of multiple extruders (or other processors) is that products can be separated prior to the next stage. Alternatively, the extruder can be a single extruder with several stages; for example, four controllable sections. In some embodiments, the extruder operates in the absence of a die.

Pressure may be increased by a feed pump. A heat exchange fluid flowing around the extruder and/or through the auger may be used to maintain temperature. The extruder screw can be temperature controlled with a central line for a heat exchange fluid pumped down the middle of the screw and a return flow path around the central flow path around the periphery of the screw (still internal within the screw).

Instead of an extruder, the apparatus can be described as a conical heat exchanger or a moving spiral heat exchanger. Flow is typically horizontal but other orientations are possible.

In the present invention, collagen can be isolated in a process that includes a step of pumping the raw material collagen source through an extruder with low shear (e.g., about 100 sec⁻¹) and typically at temperatures that do not exceed about 45° C., about 30° C., about 26° C., or about 25° C., and a residence time through the extruder of about 3 minutes or less, preferably 1 minute or less, and typically in the range of about 10 seconds to about 120 seconds. The process is not a batch process and produces a collagen-concentrated feed stream in a continuous manner. Although the extruder may contain an auger, the auger is not the primary mover of collagen through the extruder; rather collagen forms a stream that flows through the extruder faster than the motion of the auger. For screw augers, the product slides like a ribbon down the flight of the screw. In some preferred embodiments, the extruder has a flight pitch in the range of about 1 cm to about 5 cm, or about 2 cm to about 3.5 cm. In some configurations the screw profile has a range of 1:4 or alternatively 1.25:1. The screw profile can be changed to accommodate differing materials requiring different compression or shear rates.

Temperature at the inlet can be, for example, about 4° C. or higher, and the temperature within the extruder can be as high as about 80° C., however, a temperature of about 40 to about 60° C. may be typical and a temperature that is lower than the denaturation temperature of the collagen to be extracted (e.g., lower than about 45° C., about 30° C., about 26° C., or about 25° C.) is preferred to minimize denaturing. Typically, the product stream increases in temperature profile about 4 to about 20° C. (for example from about 35° C. at the inlet to about 39 or 45° C. at the outlet). Typically, the external wall of the screw is warmer than the inner wall of the extruder so that a temperature gradient is present with the material near the exterior at a higher temperature than the material nearest the screw.

Pressure is typically increased down the extruder, and the diameter of the conduit through the extruder may decrease from about 50% to about 85%, preferably about 75%, for example from about 1 inch diameter down to about 0.25 inch at the exit. Oils will be squeezed out from the feed material and can form a boundary layer that protects the collagen feed material from friction—resulting in less denaturation; the extracted fat can act as lubrication. The final section of the extruder is typically the warmest and the material under compression has the highest velocity.

In addition, or alternatively, the continuous process can be described as a multi-stage process to facilitate the temperature, pressure and shear modulated separation of the components of the raw material (typically animal skins, such as fish skins). This can be achieved by gradual heating of the skin (for example by staged heating) without a steep temperature gradient between any apparatus surface and the product. In preferred embodiments the maximum temperature difference between a surface and the adjacent product is not more than about 20° C. and ideally about 5° C. Pressure may be modulated by a pump at the outlet (typically in conjunction with a pump at the inlet) and/or use of a valve at the outlet. The shear profile is modulated by the gap between the flight and the wall and/or the speed of rotation of the rotor. Another related factor is the heat exchange; higher rates of rotor speed produce better heat transfer; greater turbulence enhances heat transfer. Pump speed, screw speed and temperature can be controlled as a function of distance down the processor to regimes appropriate for improving collagen extraction yield while preserving triple helical content and avoiding fragmentation and denaturation, or for promoting denaturation of collagen, depending on the desired collagen product profile.

### Separation Technology and Post Processing

Product exiting the processor (e.g., extractor or extruder) as a collagen-containing liquid fraction may be subjected to one or more separation steps such as filtration or passage through a membrane. An example separation process utilizes centrifugal force, such as in a continuous fashion. Separation may be conducted via gravity. For example, a triple decanter centrifuge can be used to obtain the purified collagen. Additional purification steps can be used to obtain still greater product purity. The other, non-collagen, components (e.g., lipids, oils, fats, fatty acids and esterified fats, skin (e.g., marine skin) mucous, and muscinylated extracts, such as skin (e.g., fish skin) peptides and other feed stock proteins and peptides) may be collected and used or incorporated into other useful products.

### Compositions

Collagen (isolated collagen) and collagen-containing compositions are provided herein. In some embodiments, any of at least about 5%, 10%, 15%, 20%, 25%, 30%, 35%, 40%, 45%, 50%, 55%, 60%, 65%, 70%, 75%, 80%, 85%, 90%, or 95% of the collagen is in a nondenatured form, such as triple helical collagen with molecular weight greater than 220 kDa. In some embodiments, the majority of the collagen, i.e., greater than any of about 50%, 55%, 60%, 65%, 70%, 75%, 80%, 85%, 90%, or 95% is nondenatured. In some embodiments, any of at least about 5%, 10%, 15%, 20%, 25%, 30%, 35%, 40%, 45%, 50%, 55%, 60%, 65%, 70%, 75%, 80%, 85%, 90%, or 95% is denatured (e.g., gelatin). In some embodiments, the collagen may include a combination of denatured and nondenatured forms in any ratio.

In some embodiments, the extracted collagen (isolated collagen) or collagen-containing composition may be prepared via any of the methods described herein.

An isolated collagen or composition thereof as described herein, or an extracted collagen-containing composition or a collagen-containing liquid prepared as described herein (e.g., nondenatured collagen, denatured collagen, or combinations thereof), may be used as or incorporated into a composition or article, such as but not limited to, a food, nutritional supplement, nutraceutical, animal feed, pharmaceutical dosing, drug delivery or gene carrier formulation, wound or burn care dressing, salve, ointment, lotion, or droplet formulation, cosmetic, cosmeceutical, collagen scaffold, cultivated meat or meat analogue, cell culture carrier or medium, additive manufacturing 3-D printable matrix, 3-D bio-ink for tissue engineering, cell or organ culture, and/or as a medical device component, or protein-based container, carrier or packaging material.

### Applications of Use

Collagen as described herein, such as isolated collagen, or collagen that is extracted according to any of the methods described herein (e.g., nondenatured collagen, denatured collagen, or combinations thereof) may be included in numerous methods and applications of use, including but not limited to: advanced cell culture materials and matrices; 3-D printable bio-inks for tissue engineering devices with new rheological properties; wound coverings and wound healing; scaffolds for regenerative medicine and reconstructive surgery; topical dressings, salves, ointments, lotions, creams; cosmetic ingredients to dermal enhancement/repair/beautification; plastic surgical fillers and tissue augmentation biomaterials; hemostatic agents; supporting matrices for synthetic edible meats and protein food products; components of synthetic dermal and partial and full thickness skin replacements or transient coverings; injectable tissue fillers and bulking agents; 3-D printable bio-inks for creating synthetic protein edible products and meats with novel leptic, flavor and fragrance enhancements, and textural properties; pharmaceutical delivery devices and carriers; edible and ingestible film; degradable (e.g., biodegradable) packaging materials; and as a functional food ingredient, e.g., as a rheology and structural modifier, preservative, anti-oxidant, and/or nutritional enhancer.

### EXAMPLES

The following examples are intended to illustrate, but not limit, the invention.

### Example 1—Catfish Skin PEF Pretreatment to Facilitate Screw Press Mechanical Extraction of Collagen

Catfish skin comprises two layers: the outer epidermis and the inner dermis (or corium) which differ in composition, structure and function. The outer layer epidermis is comprised of a multi-layer epithelium, including specialized cells such as the surface mucosal cells which are known to contain biologically active macromolecules, predominantly glyco-proteins important in biological functions such as immune function. The inner dermis consists of a layer of dense vascular connective tissue composed of a complicated network of cells such as lymphocytes, fibroblasts and monocytes, fibrous material such as collagen and elastin and various macromolecules synthesized by the cells. The skin collagen exists as both fibrous protein and protein sheets—depending on its function (“Skin Characteristics of Pangasius Catfish in Indonesia,” N. H. Sadi & G. P. Yoga, International Conference on the Ocean and Earth Sciences, 2021). Catfish skin extraction processes can exploit this unique collagen histology.

PEF treatment was conducted using a PEF Pilot Dual machine (Elea Technology GmbH, Germany) on fresh catfish skins or frozen fresh catfish skins. Just prior to testing, both frozen fresh and fresh catfish skins were refrigerated at 4° C. then briefly warmed to 26° C. 500 g of each skins were transferred to individual PEF treatment cells, each covered with 200 mL water and conductivity measured to be around 1000 μS/cm for each sample. The treatment cell was then placed in the PEF machine and treated. Following PEF treatment, the skins were cut into pieces sized to be about 1 to 2 inches (2.5 to 5 cm) and the pieces were fed through a conical screw press attachment on a KitchenAid mixer. The fish collagenous material passed through the screw press was collected and frozen for shipment to an analytical laboratory. The pressed skins not passing through the screw press screen were also collected and placed on wire racks. For the control samples, these skins were rinsed with tap water and drained.

All PEF treatments were conducted at a field strength of 3 kV/cm. Other conditions were: machine voltage 24 kV, electrode distance 8 cm, frequency 20 Hz, pulse width 6 μs, average energy pulse about 60J/pulse. The applied energy was varied among samples with testing at: 10, 20, 40, and 96 kJ/kg, with the controls at 0 kJ.

Pressing was observed to yield substantially more liquid from the fresh catfish skins than from the frozen skins. For the samples treated at highest energy levels (96 kJ/kg), the skin itself, rather than just liquid and fatty tissue, passed though the press screen. Yield based on mass of lyophilized dried material that passed through the screen is shown in Table I below. Collagen content was calculated from hydroxyproline content using the factor F=7.46 specific for bovine skin. Nonetheless, this factor might not be applicable to fish skin (i.e., other factor numbers are variable in published literature as no generally accepted factor for fish collagen calculation exists). Total protein content was calculated from total nitrogen content analysis using the factor F=6.25 suitable for determining fish protein.

TABLE I
PEF Treatment Facilitates Collagen
Screw Extraction from Fish Skin

| Applied Energy | Yield of bulk collagenous material collected |
|---|---|
| | Fresh Skins |
| none (control) | 14% |
| PEF 10 kJ/kg | 13% |
| PEF 20 kJ/kg | 15% |
| PEF 40 kJ/kg | 19% |
| PEF 96 kJ/kg | 18% |
| | Frozen skins |
| none (control) | 7% |
| PEF 40 kJ/kg | 10% |

Collagenous viscous material passing through the conical screw press screen was sent to an analytical laboratory and analyzed for hydroxyproline (collagen). For the control sample (no PEF), the amount of collagen was less than 0.48% which is considered undetectable, 91% water and 3.6% protein (all by mass). Similar results to control were observed for the 40 kJ/kg PEF sample (collagen-undetectable, 89% water, 5.1% protein). Surprisingly, for the sample treated with 96 kJ/kg PEF, the sample pressed through the screen contained 1.68% collagen, 85% water, 6.6% protein.

Table II shows results from another test run for the identical PEF (3 kV/cm)/screw extrusion process with temperature controlled to 27° C. and applied energy of 96 kJ/kg on frozen fresh catfish skins.

TABLE II
PEF Treatment Facilitates Collagen Screw
Extraction from Frozen Fish Skin

| | Yield of bulk collagenous material collected |
|---|---|
| Applied Energy | from frozen skins |
| none (control) | 12% |
| PEF 96 kJ/kg | 19.7% |

Collagenous viscous material passing through the conical screw press screen was sent to an analytical laboratory and analyzed for hydroxyproline (collagen). Table III shows analytical results comparing the control frozen fresh fish skin samples: no PEF pre-treatment versus PEF-treated frozen fresh fish skin.

TABLE III
PEF Treatment (96 kJ/kg) Enhances Collagen Content
in Screw Extraction from Frozen Fish Skin

| | No PEF (control) | PEF treatment (96 kJ/kg) |
|---|---|---|
| Hydroxyproline content | 0.2 | 1.4 |
| (% dry weight) | | |
| Collagen content | 1.5 | 10.4 |
| (% dry weight) | | |
| Protein content | 20.6 | 32.5 |
| (% dry weight) | | |
| Fat content | 78 | 67.9 |
| (% dry weight) | | |

PEF pre-treatment exhibited an energy-dependent enhancement of both total protein and selective collagen extraction from catfish skins in screw extrusion mechanical processing compared to no-PEF treatment. This analysis showed the unexpected results of selective collagen extraction using PEF in combination with mechanical extraction. Additionally, non-thermal PEF treatment energy input levels were important to this extraction effect: both untreated and low level (1-40 kJ/kg) PEF treatments extracted up 20.6% protein while the highest-level PEF (96 kJ/kg energy) extractant yielded 32.5% protein. The dramatic change in this protein extraction yield was attributed to the greater PEF-mediated collagen mechanical extraction.

In the untreated/low level PEF energy treatment (i.e., 10-40 kJ/kg), the extracted collagen level was 1.5%, while in the PEF highest energy level (96 kJ/kg) treatment extrudate, the collagen was 10.4% of dry material. This dramatic improvement of selective protein extraction from fish skin as collagen is unique to this new disclosed broad process of mechanical extraction.

### Example 2—Catfish Skin Collagen Extraction by Mechanical Extrusion

Freshly harvested/killed and gutted catfish are refrigerated at 4° C. for up to 36 hours. The skin (epidermal layer, including the collagen and connective tissue) is removed using a commercial de-skinner, such as a Townsend brand fish skinner—manufactured by Marel industries, or a rotating knife, or by hand, in order to leave the intact fish filets for further processing and use. Preferably, the skins are immediately chilled to 4° C. with ice or chilled water to minimize bacterial growth and enzymatic activity. The harvested skins can typically range in length from only an inch or two up to about 12 inches depending on the size of fish and the capability of the de-skinning method, and are typically 0.5-2 millimeters in thickness depending on the age of the fish, and it is desirable to control the size of the skins to 25-100 mm×25-100 mm by either cutting by hand using a sharp knife or by using a commercial size reduction equipment such as a Holac dicer/slicer which provides a clean-cut with minimal shear or distortion of the skins during cutting, thereby maintaining the quality of the intact skin tissue. The ideal size is dependent on the scale of the collagen extraction equipment. In this example, the majority of the skins are cut to approximately 60×60 mm squares (+/−20 mm). The tighter the conformance to the desired target size of cut skin pieces, this may better enable the optimal separation of the collagen from the remaining skin/epidermal structure and lead to less contamination of the collagen extract with other tissue containing material.

For collagen extraction, the chopped and sized catfish skins are fed into a 2.25-inch diameter Bonnot low shear cooker-extruder using a positive displacement pump such as Vemag or Moyno pump connected to the extruder with a 3-inch ID stainless steel pipe in a sealed system, such that the pump setting determines the flow-rate of the chopped catfish skins through the downstream collagen extraction in the extruder. The Bonnot extruder is equipped with a variable speed drive with a range of 25-175 RPM. The barrel and screw of the extruder are equipped for heating and cooling within a range of 4-100° C. Higher or lower temperatures can be achieved using alternative heating media. In this example, the chopped catfish skins are processed through the extruder and exit with a bulk temperature less than 30° C., with a target bulk exit temperature of 25° C. The desired temperature will vary depending on the starting raw material. For instance, poultry skin or pork skin will require higher energy inputs to achieve separation. The internal pressure in the extruder is controlled to not exceed 50 psi, with an ideal operating pressure of 10-20 psi. As stated previously, the flow-rate of the chopped catfish skins in the collagen extraction (executed in the extruder) is controlled by the positive displacement pump feeding the skins to the extruder, and the level of shear to which the skins are exposed within the extruder is controlled by the rotational screw speed, and this may be adjusted and optimized with the flow-rate. In this example, the extruder screw is 1:4 compression ratio. The extruder itself has an 8:1 L:D ratio. Moderate shear rates (100-300 sec⁻¹) and shear stress (about 20-40 kPa) are applied to fish skins in one embodiment.

### Example 3—Catfish Skin Collagen Extraction by Hydraulic Compression

Catfish skins are prepared as described in Example 2. The size reduction step is identical, with desired piece size being approximately 60×60 mm. The skins are placed in the filling chamber of the Protecon MRS 10 perforated compression cylinder (or similar model). The cylinder is charged with approximately 2.5 kg of skins for optimal separation. The compression cylinder is cooled to minimize adiabatic heating of the material to be compressed. The pressing time is from 10-30 seconds with maximum pressures ranging from 14-250 psi. The piston speed is varied to control both pressure and shear, and to optimize the efficiency of separation. Screen sizes are adjusted to improve separation of collagenous extrudate and to minimize passage of the epidermal material. The extracted collagen materials are passed through a screen size ranging from 0.5 mm to 1.5 mm and the compressed catfish epidermal material is retained for further processing.

### Example 4—PEF Pre-treatment in Combination with Collagen Hydraulic Pressing Extraction

Both fresh and frozen catfish skins were pre-treated using a PEF Pilot Dual machine (Elea Technology GmbH, Germany) using PEF equipment and conditions identical to Example 1 above (3 kV/cm, 40 kJ/kg). The catfish skin was reduced in size (12×12×2 mm) in order to accommodate the pilot scale test equipment. The skins (500 g, either fresh or frozen) were placed in the PEF test cells and covered with sufficient water (200 ml) to eliminate air pockets in the raw materials stream that could interfere with effective treatment. The skins were exposed to a series of conditions varying field strength (kV/cm) and total energy input (kJ/kg) to produce collagen extraction. The treated samples were then processed through a hydraulic press (mesh size 1-2 mm) to separate the liquid phase suspended collagenous materials from the residual epidermal solid structure retained. Samples of liquid extrudate and residual retained material were flash fresh frozen and lyophilized (0.11 mbar and −46° C.) for analysis. PEF pre-treatment produced 16.5% solids yield in hydraulic pressing, while hydraulic pressing alone without PEF treatment produced 12.5% solids yield, demonstrating a significant effect of PEF pre-treatment in extracting raw fish skin materials.

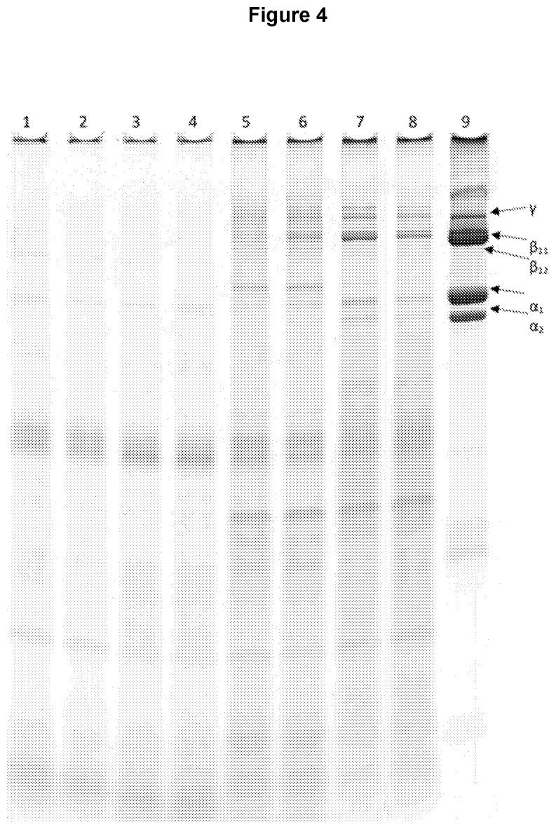

FIG. 4 compares the SDS-PAGE denaturing gel electrophoresis stained image results for identical sample volumes (15 μL per lane) and normalized total protein amounts (7.5 μg per lane) of non-PEF treated (defatted using dichloromethane extraction) fish skin collagen samples from both hydraulic press and mechanical screw extrusion extractions (also see Example 1 Tables), compared to similarly defatted PEF-hydraulic press and PEF-mechanical screw extrusion collagen extraction (also see Example 1 Tables) samples.

The SDS-PAGE gel electrophoretic pattern characteristic for the bovine acid-soluble extract collagen standard (ASC) is shown in lane 9.

The characteristic collagen monomeric a1 and a2 chains, and the collagen dimeric β11 and β12 chains, and the collagen trimeric denatured y-chain, as well as high-molecular weight non-denatured collagen components at the top of the gel lanes, are clearly visible in all lanes, all samples, but at different gel band intensities, indicating different relative amounts in each sample as a function of their respective processing. Low energy PEF (40 kJ/kg) and non-PEF control processes extracted less collagen, as indicated by weaker gel bands observed across all possible collagen products expected. Higher energy PEF (96 kJ/kg) energy and mechanical extraction is consistent with Tables I-Ill above in Example 1 in producing highest collagen yields, evidenced by highest band intensities in those characteristic bands as well as the higher-molecular weight bands greater than 200 kDa not running in the gel (cut-off 200 kDa) at the top of lanes 7 and 8. This collagen band pattern of γ-, β- and α-chains was also found for highest energy (PEF 96 kJ/kg) treatments. However, distinctly greater fractions of collagen trimer γ- and dimer β- and reduced single denatured gelatin α-chains are observed in Lanes 7 and 8 compared to all other processes. This indicates: 1) greater collagen extraction; 2) greater preservation of fish collagen multiple-strand structure; and 3) increased fraction of higher molecular weight components in the PEF-mechanical processed samples at highest energy extraction (see Lanes 7 and 8, PEF 96 kJ/kg plus extrusion).

### Example 5—Catfish Skin Collagen Extraction in Dewatering Press

Catfish skins were prepared as described in Example 2. The size reduction step was identical, with desired piece size being approximately 60×60 mm. The skins were fed into a single screw Vincent dewatering press Model CP-4. In this example, the skins were conveyed through a tube with a slotted sleave and an internal sleave with openings of 0.094 inches to 0.023 inches.

In one example, the single screw was minimally effective in conveying the skins with minimal collagen separation.

In a second example a twin screw press TSP Series was utilized in which the catfish skins were conveyed much more effectively. The resulting collagen exudate was significantly improved.

In both trials, skin temperature was maintained at less than 20° C. using chilled water. Screw speed and moisture level were varied to facilitate the feed rate and compression level.

Samples were collected at intervals along the barrel to determine rates of collagen separation and to evaluate effectiveness of the separation. The residual epidermal material was collected at the end of the dewatering screw and retained for further processing and analysis.

### Example 6—Collagen Extraction from Poultry Skin

As with previous examples, chicken skin material was maintained at temperatures less than 4° C. to prevent spoilage, enzymatic degradation and collagen denaturation. The skin was reduced in size (60×60 mm) as described in previous examples. Size was determined based on scale of testing equipment. As in previous examples, the skins were processed using mechanical separation at various rates, temperatures and pressures (shear stresses). As poultry skin starting material has different composition than catfish skin, the ratio of fat/protein required adjustment in operating conditions to apply energies sufficient to extraction collagen effectively. Poultry fat for instance has a higher melting point and fatty acid composition than fish oils, requiring higher temperatures, pressure and shear rates in order to be mechanically extracted. The same is true for avian collagen, which is more tightly bound, requiring more energy (up to 96 kJ/kg) to achieve separation. As determined by differential scanning calorimetry, the denaturation temperature for chicken collagen occurs at a higher level, approximately 40° C. vs 30° C. for catfish. Shear rates of 100-300 sec⁻¹ and shear stresses of 35 kPa effect fat and collagen separation.

### Example 7—Collagen Extraction from Pork Skin

As with above examples, skin temperature is maintained at refrigerator temperatures, (4° C.) prior to processing to minimize spoilage and enzymatic degradation. Size of the skin tissue is reduced (approx. 60×60×10-15 mm), to optimize effectiveness of separation equipment. The composition and structure of pork skin is significantly different than chicken or fish. As collagen distribution in porcine skin is distributed in a complex interwoven network distinct from avian or marine collagens, significantly more energy is required to achieve physical mechanical extraction. To accommodate these physiologic differences, higher levels of shear (100-200 sec⁻¹), elevated pressure (300-450 psi) and elevated temperature (35-40° C.) are required in extraction processes, in order to achieve separation of collagenous materials from the epidermal materials.

### Example 8—Applications of Ultrasonic Cavitation Energy in Combination with Above Collagen Extraction Methods

Fish skin samples suspended in aqueous media are introduced to a circulating ultrasonic processor described above via the sample inlet port and transported into the ultrasonic generator chamber by the pump to receive ultrasonic energy controlled by the interfaced control unit. The ultrasonic-treated samples then are re-circulated back to the sample vessel again and are re-introduced under continuous circulation to the ultrasound-generating unit for further acoustic energy application. The process is subject to cooling to limit sample heating to a temperature below 40° C. and preferably below 30° C. for preserving triple helical collagen native content. This process continues until collagen is extracted sufficiently from the raw material. This process uses 20 kHz acoustic wave energy of 1000 W power intensity for about 2 hours but up to 4 hours at controlled temperature of 4° C. One skilled in the art will recognize that acoustic energy modulation through frequency (kHz), power (W), time (minutes-hours), and type of ultrasonic generation (e.g., plate, probe) can provide different acoustic energy conditions for such processing depending on raw material size, density and species, and product desired (e.g., denatured collagen and gelatin versus native triple helical collagen). When extracting collagen, the solution is cooled and maintained below 30° C. and preferably around 4° C. using a low-temperature water cooler to prevent temperature increases due to ultrasonic treatment.

In a second alternative ultrasound extraction pre- or post-treatment process, an aqueous slurry containing small minced fish skin fragments (˜2 mm across by 2 mm thick) are subjected to ultrasound energy using an ultrasound reactor (e.g., model Vibra-Cell, Sonics & Material, Inc., Newtown, CT, USA) and a flat tip probe (diameter 25 mm) with maximum amplitude (100%) at 70 μm of applied acoustic wave height. Extraction is carried out at 750 W at a frequency of 20 kHz in pulse mode (on/off at 5 second periods) for 30 min. During extraction and ultrasonication, the processing temperature is maintained at 4° C. using an interfaced programmable cooler.

In a third alternative ultrasound pre or post-treatment process, a 13-mm diameter ultrasonication probe is used at an amplitude of 70% with pulse mode (on/off at 10 second periods) for 10 min. The processed mixture is then hydrodynamically sheared (e.g., at 100 sec⁻¹) for 1 hour at 4-8° C.

Finally, the resulting solids and liquid extractant phase from acoustic processing are subject to further extraction of collagen using processes described herein.

### Example 9—Extraction of Catfish Gelatin and Partially Denatured Collagen Using a Thermo-Mechanical Process

Fresh catfish skins starting material is maintained at a temperature less than 4° C. to minimize spoilage and degradation. The skins are size reduced as described in previous examples, using a Holac Dicer (or equivalent) to reach a size of 60×60 mm. The skins are treated using a continuous PEF Pipe process. After PEF, the skins are pumped using a positive displacement pump into the Bonnot extruder. Unlike previous examples where temperatures are controlled to not exceed the denaturation temperature of catfish collagen, in this example the process temperatures are deliberately elevated to exceed the minimum collagen denaturation temperature, in order to extract gelatin and denatured collagen. By necessity, heating of the material in excess of 40° C. through active heating or PEF-induced heating achieves denaturation and partial disassembly of the collagen triple helix structure to release a mixture of undenatured collagen and gelatin under mechanical means in a shortened 6-hour process.

### Example 10—Supercritical Fluid Extraction of Fats and Lipids from Collagen

Commercial SFE equipment (e.g., HighTech Extracts, Biddeford, ME, USA, Dual 7L Extraction System) with adjustable extraction supercritical CO₂ parameters of pressure (P), temperature (T), extraction time (Tₑₓₜ), and flow rate (F) is deployed for extraction of fats and lipids. Co-solvents water and ethanol can be introduced to improve extraction media polarity and efficiency for process optimization. These SFE parameters can be varied by one skilled in the art along routine parameters to extract fats and lipids from animal sources of collagen by pre-treatment or process-extracted de-watered or dried collagen solids post-treatment.

As pre-treatment SFE, dewatered or dried animal raw materials solids as small pieces (e.g., 2 mm-cubes) are loaded into the SFE extraction chambers and sealed into the SFE instrument. As post-treatment SFE, dewatered or dried extracted collagen or gelatin products as small pieces or fragments (e.g., 2 mm-cubes) are loaded into the SFE extraction chambers and sealed into the SFE instrument. In both cases, the automated SFE process for CO₂ or CO₂ and a co-solvent (e.g., water) are produced by pumps and thermal controllers.

The SFE compressed CO₂-based solvent mixture can have a temperature of about 0° to about 33° C. and a pressure of about 800 to about 5000 psi. As such, the SFE compressed CO₂-based mixture can be a liquid or a supercritical fluid, depending on the media, temperature and pressure selected. For example: P: 10.3-25 MPa; T: 25-35° C., Tₑₓₜ: 50-150 min; F: 3-50 g/min CO₂ can be applied to minced de-watered or dried fish skin samples of −2 mm cubic piece size and shape. In another embodiment, P=45 MPa, T=40° C., F=6 kg/h CO₂, and Tₑₓₜ=120 min. Addition of a co-solvent water or ethanol (5-15% by mass) to supercritical CO₂ can be used to enhance the SFE efficiency at the same flow rates (e.g., 6 kg/h), as long as SFE conditions are maintained for liquid CO₂ and the SFE extraction temperature is controlled below 36° C. and preferably below 32° C. (e.g., P=25 MPa; F=6 kg/h CO₂; T=34° C.; Tₑₓₜ=300 min). Furthermore, FDA-approved GRAS (as listed here: https://www.ecfr.gov/current/title-21/chapter-I/subchapter-B/part-182) emulsifiers or food-grade surfactants (preferably known GRAS ethoxylated emulsifier/surfactants such as Poloxamers, Pluronics or Tweens) can be added to the co-solvent with CO₂ majority fraction to enhance the SFE processing by forming supercritical micellar phases, as long as supercritical temperature is controlled below 36° C. and preferably 32° C.

Although the foregoing invention has been described in some detail by way of illustration and examples for purposes of clarity of understanding, it will be apparent to those skilled in the art that certain changes and modifications may be practiced without departing from the spirit, intent, and scope of the invention, which is delineated in the appended claims. Therefore, the description should not be construed as limiting the scope of the invention.

All publications, patents, and patent applications cited herein are hereby incorporated by reference in their entireties for all purposes and to the same extent as if each individual publication, patent, or patent application were specifically and individually indicated to be so incorporated by reference.

We claim:

1. A method for extracting collagen from isolated animal tissue, said method comprising:
non-thermal mechanically and/or hydrodynamically shearing an animal tissue that comprises collagen in an aqueous media, thereby separating a collagen-containing liquid fraction comprising extracted collagen from a solid fraction, wherein the shearing is conducted under controlled conditions of temperature, pressure, and hydrodynamic energy such that at least about 5% of the extracted collagen in the collagen-containing liquid fraction is in a nondenatured form.

2. The method of claim 1, wherein greater than about 50% of the extracted collagen in the collagen-containing liquid fraction is in the nondenatured form.

3. The method of claim 1, wherein the extracted nondenatured collagen comprises a molecular weight greater than about 220 kDa.

4. The method of claim 1, wherein at least about 5% of the extracted collagen in the collagen-containing liquid is in a denatured or hydrolyzed form.

5. The method of claim 1, wherein greater than about 50% of the collagen in the collagen-containing liquid is in the denatured or hydrolyzed form.

6. The method of claim 1, wherein the collagen-containing liquid fraction comprises greater than about 5% of the collagen content in the isolated animal tissue from which it is extracted.

7. The method of claim 1, wherein said isolated animal tissue is maintained at a temperature that is less than about 10° C. prior to mechanical and/or hydrodynamic shearing.

8. The method of claim 7, wherein microbial or enzymatic degradation of collagen or activation of endotoxins is limited in comparison to an isolated animal tissue raw material that is not maintained at a temperature less than about 10° C.

9. The method of claim 1, wherein said mechanical and/or hydrodynamic shearing of the isolated animal tissue that comprises collagen comprises one or more of:
(i) low shear extrusion, optionally at about 100 sec⁻¹ or less, through an extruder that comprises an inlet, a compression screw, a barrel, and a positive displacement pump, wherein the tissue is pumped through the barrel with the compression screw under pressure of about 10 psi to about 150 psi and with temperature conditions controlled such that the difference in temperature between the inlet and outlet does not vary more than about 20° C., wherein extruded material that flows through the outlet is further separated into the collagen-containing liquid fraction that comprises extracted collagen and the solid fraction;
(ii) compression in a perforated compression cylinder, wherein the cylinder is compressed at a pressure of about 10 psi to about 150 psi and a cycle time of about 5 sec to about 30 sec, wherein the collagen-containing liquid fraction flows through perforations on the periphery of the cylinder; or
(iii) processing through a dewatering press apparatus at a pressure of about 10 psi to about 150 psi and a residence de-watering time of about 5 sec to about 30 sec.

10. The method of claim 9, wherein the collagen-containing liquid fraction and/or the solid fraction is further processed under the same or different conditions in the same or different apparatus to improve yield and/or purity of the extracted collagen.

11. The method of claim 1, wherein prior to shearing the isolated animal tissue, said tissue is subjected to an input of electric field and/or acoustic energy that does not substantially degrade the structure of the collagen extracted from the tissue.

12. The method of claim 11, wherein the input energy is provided by pulsed electric field and/or ultrasonication treatment.

13. The method of claim 11, wherein the input of energy comprises pulsed electric field treatment at an electric field strength that is about 3 kV/cm to about 90 kV/cm.

14. The method of claim 11, wherein the input of energy comprises ultrasonic treatment at about 20 Hz to about 40 Hz and about 150 W to about 250 W instrument input power.

15. The method of claim 11, wherein the input of energy comprises about 40 kJ/kg to about 100 kJ/kg of the isolated animal tissue.

16. The method of claim 11, wherein the input of energy does not raise the temperature of the isolated animal tissue or a processing milieu in which it resides above about 30° C.

17. The method of claim 1, wherein prior to mechanical and/or hydrodynamic shearing the tissue, the isolated animal tissue is mechanically diced, macerated, minced or ground, thereby processing the tissue into smaller pieces under low shear conditions.

18. The method of claim 17, wherein the isolated animal tissue is mechanically diced or minced into pieces that comprise an average cross-sectional area of about 0.5 square inches to about 2 square inches.

19. The method of claim 11, wherein prior to the input of energy, the isolated animal tissue is mechanically diced or ground into smaller pieces under low shear conditions.

20. The method of claim 19, wherein the isolated animal tissue is mechanically diced or minced into pieces that comprise an average cross-sectional are of about 0.5 square inches to about 2 square inches.

21. The method of claim 1, wherein said isolated animal tissue comprises skin tissue.

22. The method of claim 21, wherein the skin tissue comprises fish skin.

23. The method of claim 1, wherein the collagen-containing liquid fraction is produced from the isolated animal tissue in less than about 6 hours.

24. The method of any of claims 1 to 23, wherein the method does not comprise energy, chemical, and/or enzymatic conditions that intentionally denature or hydrolyze collagen.

25. The method of claim 24, wherein the method does not include conditions that intentionally degrade or hydrolyze collagen to produce fragments that are smaller than about 220 kDa.

26. The method of any of claims 1 to 23, wherein at least about 90% of the method is conducted at a temperature that is lower than the denaturation temperature for the collagen.

27. The method of claim 26, wherein at least about 95% of the method is conducted at a temperature that does not exceed 30° C.

28. The method of any of claims 1 to 23, wherein at least about 70% of the method is conducted at a mechanical shear rate that is below 100 sec⁻¹.

29. The method of any of claims 1 to 23, wherein the collagen-containing liquid fraction comprises an aerobic plate count for microbial contamination less than about 104 CFU/g.

30. The method of any of claims 1 to 23, wherein the collagen-containing liquid fraction comprises at least about 10²-fold reduction in aerobic plate count for microbial contamination in comparison to the isolated animal tissue from which it was extracted.

31. A collagen-containing liquid prepared by the method of any of claims 1 to 23, wherein at least about 5% of the extracted collagen is in the nondenatured form.

32. The collagen-containing liquid according to claim 31, wherein greater than about 50% of the extracted collagen is in the nondenatured form.

33. A composition comprising isolated collagen, wherein said isolated collagen is extracted collagen that is purified from the collagen-containing liquid according to claim 31.

34. The composition of claim 33, wherein the extracted collagen comprises a molecular weight greater than about 220 kDa.

35. A composition comprising isolated collagen, wherein greater than about 50% of said collagen is in a nondenatured form.

36. The composition of claim 35, wherein the nondenatured collagen comprises a molecular weight greater than about 220 kDa.

37. A food, nutritional supplement, nutraceutical, animal feed, pharmaceutical dosing, drug delivery or gene carrier formulation, wound or burn care dressing, cosmetic additive in a gel, cream, salve, drop, ointment or topical dressing, cosmeceutical, collagen cell culture scaffold, cultivated meat or meat analogue, additive manufacturing 3-D printable matrix, 3-D bio-ink for tissue engineering, cell carrier, and/or medical device component, or container or packaging material, comprising a composition according to claim 33 or 35.

38. A food, nutritional supplement, nutraceutical, animal feed, pharmaceutical dosing, drug delivery or gene carrier formulation, wound or burn care dressing, cosmetic additive in a gel, cream, salve, drop, ointment or topical dressing, cosmeceutical, collagen cell culture scaffold, cultivated meat or meat analogue, additive manufacturing 3-D printable matrix, 3-D bio-ink for tissue engineering, cell carrier, and/or medical device component, or container or packaging material, comprising a collagen-containing liquid according to claim 32.

39. A method for extracting collagen from isolated animal tissue, said method comprising:
mechanically and/or hydrodynamically shearing an animal tissue that comprises collagen in an aqueous media, thereby separating a collagen-containing liquid fraction comprising extracted collagen from a solid fraction, wherein the shearing is conducted under controlled conditions of temperature, pressure, and hydrodynamic energy such that at least about 5% of the extracted collagen in the collagen-containing liquid fraction is in a denatured form.

40. The method of claim 39, wherein greater than about 50% of the extracted collagen in the collagen-containing liquid fraction is in the denatured form.

41. The method of claim 39, wherein said mechanical and/or hydrodynamic shearing of the isolated animal tissue that comprises collagen comprises one or more of:
(i) low shear extrusion, optionally at about 100 sec⁻¹ or less, through an extruder that comprises an inlet, a compression screw, a barrel, and a positive displacement pump, wherein the tissue is pumped through the barrel with the compression screw under pressure of about 10 psi to about 150 psi and temperature about 40° C. to about 60° C., wherein extruded material that flows through the outlet is further separated into the collagen-containing liquid fraction that comprises extracted collagen and the solid fraction;
(ii) compression in a perforated compression cylinder, wherein the cylinder is compressed at a pressure of about 10 psi to about 150 psi, temperature about 40° C. to about 60° C., and a cycle time of about 5 sec to about 30 sec, wherein the collagen-containing liquid fraction flows through perforations on the periphery of the cylinder; or
(iii) processing through a dewatering press apparatus at a pressure of about 10 psi to about 150 psi, temperature about 40° C. to about 60° C., and a residence de-watering time of about 5 sec to about 30 sec.

42. The method of claim 39, wherein prior to shearing the isolated animal tissue, said tissue is subjected to an input of electric field and/or acoustic energy.

43. The method of claim 42, wherein the input energy is provided by pulsed electric field and/or ultrasonication treatment.

44. The method of claim 42, wherein the input of energy comprises pulsed electric field treatment at an electric field strength that is about 3 kV/cm to about 90 kV/cm.

45. The method of claim 42, wherein the input of energy comprises ultrasonic treatment at about 20 Hz to about 40 Hz and about 150 W to about 250 W instrument input power.

46. The method of claim 42, wherein the input of energy comprises about 40 kJ/kg to about 100 kJ/kg of the isolated animal tissue.

47. The method of claim 39, wherein prior to mechanical and/or hydrodynamic shearing the tissue, the isolated animal tissue is mechanically diced, macerated, minced or ground, thereby processing the tissue into smaller pieces under low shear conditions.

48. The method of claim 47, wherein the isolated animal tissue is mechanically diced or minced into pieces that comprise an average cross-sectional area of about 0.5 square inches to about 2 square inches.

49. The method of claim 42, wherein prior to the input of energy, the isolated animal tissue is mechanically diced or ground into smaller pieces under low shear conditions.

50. The method of claim 49, wherein the isolated animal tissue is mechanically diced or minced into pieces that comprise an average cross-sectional are of about 0.5 square inches to about 2 square inches.

51. The method of claim 39, wherein said isolated animal tissue comprises skin tissue.

52. The method of claim 51, wherein the skin tissue comprises fish skin.

53. The method of claim 39, wherein the collagen-containing liquid fraction is produced from the isolated animal tissue in less than about 6 hours.

54. A collagen-containing liquid prepared by the method of any of claims 39 to 53, wherein at least about 5% of the extracted collagen is in the denatured form.

55. The collagen-containing liquid according to claim 53, wherein greater than about 50% of the extracted collagen is in the denatured form.

56. A food, nutritional supplement, nutraceutical, animal feed, pharmaceutical dosing, drug delivery or gene carrier formulation, wound or burn care dressing, cosmetic additive in a gel, cream, salve, drop, ointment or topical dressing, cosmeceutical, collagen cell culture scaffold, cultivated meat or meat analogue, additive manufacturing 3-D printable matrix, 3-D bio-ink for tissue engineering, cell carrier, and/or medical device component, or container or packaging material, comprising a collagen-containing liquid according to claim 54.
